SEQUENCE LISTING (37 sequences)

SEQ ID NO: 1 - protein, 268 aa, synthetic construct
  REGION 1..268: note = D3R/4-1BB chimeric TNFR
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQIISFFLALTSTALLFLLFFLTLRFSVVKRGRKKLLYIFKQPFMRPVQTTQEEDGCSCRFPEEEEGGCEL

SEQ ID NO: 2 - protein, 271 aa, synthetic construct
  REGION 1..271: note = HVEM/4-1BB chimeric TNFR (TNF receptor)
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVIISFFLALTSTALLFLLFFLTLRFSVVKRGRKKLLYIFKQPFMRPVQTTQEEDGCSCRFPEEEEGGCEL

SEQ ID NO: 3 - protein, 260 aa, synthetic construct
  REGION 1..260: note = CD27/4-1BB chimeric TNFR
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRIISFFLALTSTALLFLLFFLTLRFSVVKRGRKKLLYIFKQPFMRPVQTTQEEDGCSCRFPEEEEGGCEL

SEQ ID NO: 4 - protein, 281 aa, synthetic construct
  REGION 1..281: note = RANK/4-1BB chimeric TNFR
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPIISFFLALTSTALLFLLFFLTLRFSVVKRGRKKLLYIFKQPFMRPVQTTQEEDGCSCRFPEEEEGGCEL

SEQ ID NO: 5 - protein, 151 aa, synthetic construct
  REGION 1..151: note = Fn14/4-1BB chimeric TNFR
MARGSLRRLLRLLVLGLWLALLRSVAGEQAPGTAPDWCSRGSSWSADLDKCMDCASCRARPHSDFCLGCAAAPPAPFRLLWPIISFFLALTSTALLFLLFFLTLRFSVVKRGRKKLLYIFKQPFMRPVQTTQEEDGCSCRFPEEEEGGCEL

SEQ ID NO: 6 - protein, 409 aa, synthetic construct
  REGION 1..409: note = CD27/DR3 chimeric TNFR
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 7 - protein, 191 aa, synthetic construct
  REGION 1..191: note = ligand binding domain of CD27
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIR

SEQ ID NO: 8 - protein, 212 aa, synthetic construct
  REGION 1..212: note = ligand binding domain of RANK
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLP

SEQ ID NO: 9 - protein, 82 aa, synthetic construct
  REGION 1..82: note = ligand binding domain of Fn14
MARGSLRRLLRLLVLGLWLALLRSVAGEQAPGTAPDWCSRGSSWSADLDKCMDCASCRARPHSDFCLGCAAAPPAPFRLLWP

SEQ ID NO: 10 - protein, 202 aa, synthetic construct
  REGION 1..202: note = ligand binding domain of HVEM
MEPPGDWGPPPWRSTPRTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWV

SEQ ID NO: 11 - protein, 199 aa, synthetic construct
  REGION 1..199: note = ligand binding domain of DR3
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQ

SEQ ID NO: 12 - protein, 42 aa, synthetic construct
  REGION 1..42: note = 4-1BB signalling domain
KRGRKKLLYIFKQPFMRPVQTTQEEDGCSCRFPEEEEGGCEL

SEQ ID NO: 13 - protein, 36 aa, synthetic construct
  REGION 1..36: note = OX40 signalling domain
RDQRLPPDAHKPPGGGSFRTPIQEEQADAHSTLAKI

SEQ ID NO: 14 - protein, 58 aa, synthetic construct
  REGION 1..58: note = GITR signalling domain
QLGLHIWQLRSQCMWPRETQLLLEVPPSTEDARSCQFPEEERGERSAEEKGRLGDLWV

SEQ ID NO: 15 - protein, 48 aa, synthetic construct
  REGION 1..48: note = CD27 signalling domain
QRRKYRSNKGESPVEPAEPCHYSCPREEEGSTIPIQEDYRKPEPACSP

SEQ ID NO: 16 - protein, 62 aa, synthetic construct
  REGION 1..62: note = CD40 signalling domain
KKVAKKPTNKAPHPKQEPQEINFPDDLPGSNTAAPVQETLHGCQPVTQEDGKESRISVQERQ

SEQ ID NO: 17 - protein, 197 aa, synthetic construct
  REGION 1..197: note = D3R signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 18 - protein, 27 aa, synthetic construct
  REGION 1..27: note = transmembrane domain sequence
FWVLVVVGGVLACYSLLVTVAFIIFWV

SEQ ID NO: 19 - protein, 27 aa, synthetic construct
  REGION 1..27: note = 4-1BB transmembrane domain
IISFFLALTSTALLFLLFFLTLRFSVV

SEQ ID NO: 20 - protein, 21 aa, synthetic construct
  REGION 1..21: note = DR3 transmembrane domain
MFWVQVLLAGLVVPLLLGATL

SEQ ID NO: 21 - protein, 21 aa, synthetic construct
  REGION 1..21: note = OX40 transmembrane domain
VAAILGLGLVLGLLGPLAILL

SEQ ID NO: 22 - protein, 21 aa, synthetic construct
  REGION 1..21: note = GITR transmembrane domain
LGWLTVVLLAVAACVLLLTSA

SEQ ID NO: 23 - protein, 21 aa, synthetic construct
  REGION 1..21: note = CD70 transmembrane domain
VLRAALVPLVAGLVICLVVCI

SEQ ID NO: 24 - protein, 22 aa, synthetic construct
  REGION 1..22: note = CD40 transmembrane domain
ALVVIPIIFGILFAILLVLVFI

SEQ ID NO: 25 - protein, 193 aa, synthetic construct
  REGION 1..193: note = CD27 sequence
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 26 - protein, 277 aa, synthetic construct
  REGION 1..277: note = CD40 sequence
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 27 - protein, 417 aa, synthetic construct
  REGION 1..417: note = D3R sequence
MEQRPRGCAAVAAALLLVLLGARAQGGTRSPRCDCAGDFHKKIGLFCCRGCPAGHYLKAPCTEPCGNSTCLVCPQDTFLAWENHHNSECARCQACDEQASQVALENCSAVADTRCGCKPGWFVECQVSQCVSSSPFYCQPCLDCGALHRHTRLLCSRRDTDCGTCLPGFYEHGDGCVSCPTSTLGSCPERCAAVCGWRQMFWVQVLLAGLVVPLLLGATLTYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 28 - protein, 283 aa, synthetic construct
  REGION 1..283: note = HVEM sequence
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 29 - protein, 616 aa, synthetic construct
  REGION 1..616: note = RANK sequence
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 30 - protein, 129 aa, synthetic construct
  REGION 1..129: note = Fn14 sequence
MARGSLRRLLRLLVLGLWLALLRSVAGEQAPGTAPCSRGSSWSADLDKCMDCASCRARPHSDFCLGCAAAPPAPFRLLWPILGGALSLTFVLGLLSGFLVWRRCRRREKFTTPIEETGGEGCPAVALIQ

SEQ ID NO: 31 - protein, 112 aa, synthetic construct
  REGION 1..112: note = CD3-zeta endodomain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 32 - protein, 368 aa, synthetic construct
  REGION 1..368: note = 4-1BB and CD3-zeta endodomains
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 33 - protein, 152 aa, synthetic construct
  REGION 1..152: note = CD28 and CD3-zeta endodomains
SKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 34 - protein, 188 aa, synthetic construct
  REGION 1..188: note = CD28, OX40 and CD3-zeta endodomains
SKRSRLLHSDYMNMTPRRPGPTRKHYQPYAPPRDFAAYRSRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 35-36 - skipped (2 entries)

SEQ ID NO: 37 - protein, 7 aa, synthetic construct
  REGION 1..7: note = consensus Tobacco Etch Virus (TEV) cleavage site
ENLYFQS

(19) United States
(12) Patent Application Publication
(10) Pub. No.: US 2026/0365715 A1
(43) Pub. Date: Nov. 19, 2026

(54) Cell

(71) Applicant: AUTOLUS LIMITED, London (GB)

(72) Inventors: Martin Pulé, London (GB); Marco Della Peruta, London (GB)

(21) Appl. No.: 19/181,085

(22) Filed: Apr. 16, 2025

Related U.S. Application Data
(63) Continuation of application No. 16/772,260, filed on Dec. 14, 2018, now Pat. No. 12,304,942.

(30) Foreign Application Priority Data
Dec. 15, 2017 (GB) 1720949.5

(51) Int. Cl. C07K 14/705; A61K 40/11; A61K 40/31; A61K 40/42; C07K 16/28; C07K 16/30; C07K 19/00; C12N 5/0783

(52) U.S. Cl. CPC C07K 14/70578; A61K 40/11; A61K 40/31; A61K 40/4204; A61K 40/4211; A61K 40/4215; A61K 40/4258; C07K 14/7051; C07K 16/30; C07K 19/00; C12N 5/0636; C07K 16/2803; C07K 2319/02; C07K 2319/32

(57) ABSTRACT

The present invention relates to a cell which comprises; (i) a chimeric antigen receptor (CAR) or a transgenic T-cell receptor (TCR); and (ii) a chimeric TNF receptor (TNFR) which comprises (a) a binding domain which is capable of binding a TNFR ligand; and (b) a TNFR signalling domain.

### CROSS-REFERENCE TO RELATED APPLICATIONS

This is a Continuation of U.S. application Ser. No. 16/772,260, filed Jun. 12, 2020, now U.S. Pat. No. 12,304,942, issued May 20, 2025, which is a National Phase of International Application No. PCT/GB2018/053629, filed Dec. 14, 2018, which claims priority to Great Britain Application No. 1720949.5, filed Dec. 15, 2017.

### INCORPORATION BY REFERENCE OF MATERIAL SUBMITTED ELECTRONICALLY

A Sequence Listing is incorporated herein by reference as part of the disclosure. The Sequence Listing is submitted as an XML file named “54184A_SeqListing.xml”, which was created on Apr. 15, 2025 and is 50,658 bytes in size.

### FIELD OF THE INVENTION

The present invention relates to an engineered cell which expresses a chimeric antigen receptor (CAR) or a transgenic T-cell receptor (TCR); and in particular to approaches to control the proliferation and survival of such cells.

### BACKGROUND TO THE INVENTION

Antigen-specific T-cells may be generated by selective expansion of peripheral blood T-cells natively specific for the target antigen. However, it is difficult and quite often impossible to select and expand large numbers of T-cells specific for most cancer antigens. Gene-therapy with integrating vectors affords a solution to this problem as transgenic expression of Chimeric Antigen Receptor (CAR) allows generation of large numbers of T-cells specific to any surface antigen by ex vivo viral vector transduction of a bulk population of peripheral blood T-cells.

TNF-family co-stimulatory molecules provide survival and expansion signals for T-cells during their ontogeny. These TNF receptors (TNFRs) signal via TNF receptor associated factor (TRAF) second messengers. However, the context of these co-stimulatory signals is critical and distinct: for a T-cell to receive such a signal, both the receptor and ligand must be expressed at the same time and at the same place. For instance, for a T-cell to receive a CD27 signal, it must express the CD27 receptor and the cognate ligand CD70 must either be expressed by the T-cell itself or by other cells it is in contact with. This is very tightly controlled: for instance, 4-1BB/4-1BBL and OX40/OX40L are both expressed during a short period after activation on CD8+ and CD4+ T-cells/antigen presenting cells only if antigen is present.

This orchestration is key to physiological immune response to a viral infection, for example. However, a CAR T-cell response does not typically involve an array of accessory immune cells, but rather the CAR T-cell has to survive in the context of a hostile microenvironment with very little immunological cues. CAR T-cell efficacy depends on proliferation and engraftment of the CAR T-cells.

Accordingly, there remains a need for approaches to improve the effectiveness of engineered cells to proliferate, survive and/or engraft in a microenvironment in which the appropriate immunological cues may not be provided.

### SUMMARY OF THE INVENTION

In a first aspect the present invention provides a cell which comprises; (i) a chimeric antigen receptor (CAR) or a transgenic T-cell receptor (TCR); and (ii) a chimeric TNF receptor (TNFR) which comprises (a) a binding domain which is capable of binding a TNFR ligand; and (b) a TNFR signalling domain.

The use of a chimeric TNFR as provided by the present invention enables the tight temporal and/or spatial control of TNFR signalling to be decoupled in order to provide improved survival signals for engineered cells, for example CAR T cells. Accordingly, the chimeric TNFR of the present invention may compensate for the lack of a complete physiological immune response in a tumour microenvironment. By way of example, the chimeric TNFR may be constructed such that the antigen-binding domain is engaged, and thus a required co-stimulatory signal induced, in the expected microenvironment in which activity of the engineered cell is required.

The antigen-binding domain of the chimeric TNFR may comprise the ligand binding domain of a TNFR. For example, the antigen-binding domain may comprise the ligand binding domain of D3R, HVEM, CD27, CD40, RANK or Fn14.

The signalling domain of the chimeric TNFR may be an activating signalling domain. For example, the signalling domain may be capable of signalling via TNFR-associated factors (TRAFs). For example, the activating signalling domain may comprise the signalling portion of the 4-1BB, OX40, or GITR endodomain.

The activating signalling domain may comprise the signalling portion of the 4-1BB endodomain.

The signalling domain may not be capable of signal 1 production in the cell. For example, the signalling domain may not comprise a CD3 endodomain. The signalling domain may not comprise a CD3zeta endodomain.

In one embodiment the chimeric TNFR is selected from D3R-4-1BB (SEQ ID NO: 1), HVEM-4-1BB (SEQ ID NO: 2), CD27-4-1BB (SEQ ID NO: 3), RANK-4-1BB (SEQ ID NO: 4), Fn14-4-1BB (SEQ ID NO: 5) and CD27-D3R (SEQ ID NO: 6) or a variant with at least 80% sequence identity to any of SEQ ID NO: 1-6.

In a further aspect, the present invention comprises a cell which comprises; (i) a chimeric antigen receptor (CAR) or a transgenic T-cell receptor (TCR); and (ii) an exogenous polynucleotide expressing a TNF receptor (TNFR) which is capable of providing a co-stimulatory to the cell following binding of a ligand to the TNFR ligand binding domain

The exogenous polynucleotide may express CD27, CD40, D3R, HVEM, RANK or Fn14.

The exogenous polynucleotide may express CD27 or CD40.

The cell may an alpha-beta T cell, a NK cell, a gamma-delta T cell, or a cytokine induced killer cell.

In another aspect the present invention provides a chimeric TNF receptor (TNFR) which comprises (a) a binding domain which is capable of binding a TNFR ligand; and (b) a TNFR signalling domain which is not capable of signal 1 production.

The signalling domain may not comprise a CD3 endodomain. The signalling domain may not comprise a CD3zeta endodomain.

The antigen-binding domain of the chimeric TNFR may not be capable of binding CD70.

In a further aspect the present invention provides a nucleic acid construct which comprises:
(i) a first nucleic acid sequence which encodes i) a chimeric antigen receptor (CAR) or a transgenic T-cell receptor (TCR); and
(ii) a second nucleic acid sequence which encodes a chimeric TNF receptor (TNFR) which comprises (a) a binding domain which is capable of binding a TNFR ligand; and (b) a TNFR signalling domain.

The second nucleic acid sequences may encode any chimeric TNFR as provided by the present invention.

The first and second nucleic acid sequences may be separated by a co-expression site.

The present invention further provides a nucleic acid sequence which encodes a chimeric TNF receptor (TNFR) according to the present invention.

In another aspect the present invention provides a kit of nucleic acid sequences comprising:
(i) a first nucleic acid sequence which encodes i) a chimeric antigen receptor (CAR) or a transgenic T-cell receptor (TCR); and
(ii) a second nucleic acid sequence which encodes a chimeric TNF receptor (TNFR) which comprises (a) a binding domain which is capable of binding a TNFR ligand; and (b) a TNFR signalling domain. The chimeric TNFR may be any chimeric TNFR as provided by the present invention.

The present invention also provides a vector which comprises a nucleic acid construct according to the present invention.

In another aspect the present invention provides a kit of vectors which comprises:
(i) a first vector which comprises a nucleic acid sequence which encodes i) a chimeric antigen receptor (CAR) or a transgenic T-cell receptor (TCR); and
(ii) a second vector which comprises a nucleic acid sequence which encodes a chimeric TNF receptor (TNFR) which comprises (a) a binding domain which is capable of binding a TNFR ligand; and (b) a TNFR signalling domain as defined by the present invention.

In another aspect the present invention provides a pharmaceutical composition which comprises a plurality of cells, a nucleic acid construct, a first nucleic acid sequence and a second nucleic acid sequence; a vector or a first and a second vector according to the present invention.

In a further aspect the present invention provides a pharmaceutical composition according to the invention for use in treating and/or preventing a disease.

In another aspect the present invention relates to a method for treating and/or preventing a disease, which comprises the step of administering a pharmaceutical composition according to the invention to a subject in need thereof.

The method may comprise the following steps:
(i) isolation of a cell containing sample;
(ii) transduction or transfection of the cell with a nucleic acid construct, a vector or a first and a second vector according to the present invention; and
(iii) administering the cells from (ii) to a subject.

The cell may autologous. The cell may be allogenic.

In another aspect the present invention relates to the use of a pharmaceutical composition according to the invention in the manufacture of a medicament for the treatment and/or prevention of a disease.

The disease may be cancer.

In another aspect the present invention relates to a method for making a cell according to the present invention, which comprises the step of introducing: a nucleic acid construct, a first nucleic acid sequence and a second nucleic acid sequence, a vector or a first and a second vector of the present invention into the cell.

The cell may be from a sample isolated from a subject.

### BRIEF DESCRIPTION OF THE FIGURES

FIG. 1-a) Schematic diagram illustrating a classical CAR. (b) to (d): Different generations and permutations of CAR endodomains: (b) initial designs transmitted ITAM signals alone through FcεR1-γ or CD3ζ endodomain, while later designs transmitted additional (c) one or (d) two co-stimulatory signals in the same compound endodomain.

FIG. 2—Schematic diagram illustrating a TNFR

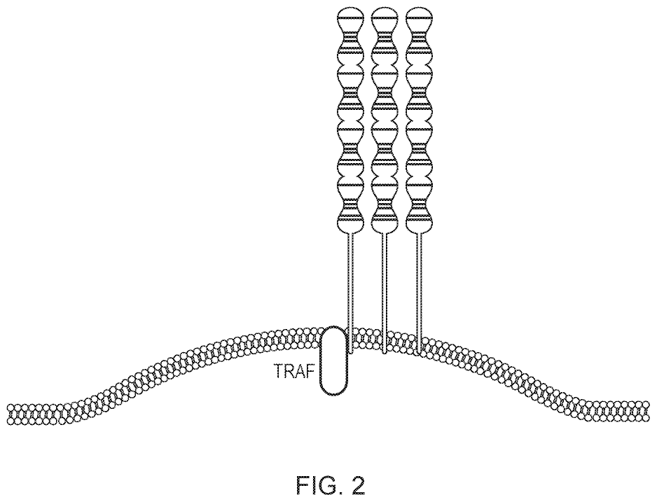

FIG. 3—Summary of the TNF superfamily

FIGS. 4A and 4B—Comparing the activity of cells expressing a GD2 CAR, with and without co-expression of a HVEM-4-1BB chimeric TNF receptor, in the presence or absence of cells presenting the ligand LIGHT, and in the presence of cells expressing the target antigen or not expressing the target antigen: a) proliferation; b) cytotoxicity against target cells.

FIGS. 5A and 5B—Comparing the activity of cells expressing a GD2 CAR, with and without co-expression of a RANK-4-1BB chimeric TNF receptor, in the presence or absence of cells presenting the ligand RANKL, and in the presence of cells expressing the target antigen or not expressing the target antigen: a) proliferation; b) cytotoxicity against target cells.

### DETAILED DESCRIPTION OF THE INVENTION

### Chimeric TNF Receptor (TNFR)

In a first aspect the present invention provides a cell which comprises; (i) a chimeric antigen receptor (CAR) or a transgenic T-cell receptor (TCR); and (ii) a chimeric TNF receptor (TNFR) which comprises (a) a binding domain which is capable of binding a TNFR ligand; and (b) a TNFR signalling domain.

As used herein, a chimeric TNFR refers to a TNFR which comprises (a) a binding domain which is capable of binding a TNFR ligand of a first TNFR; and (b) a TNFR signalling domain of a second TNFR.

Thus, following engagement by a TNFR ligand which typically binds to the first TNFR, the chimeric TNFR of the present invention is capable of transmitting the co-stimulatory signal typically provided by a second TNFR. This enables the tight temporal and/or spatial control of TNFR signalling to be decoupled in order to provide improved survival signals for engineered cells, for example CAR T cells.

By way of example, the chimeric TNFR of the present invention may comprise a binding domain which is capable of binding to a TNFR ligand such as CD70, RANL or TWEAK and the activating signalling domain of a different TNFR such as 41-BB or OX40.

Such chimeric TNFRs may be useful in target certain cancers by coupling the recognition of a TNFR ligand which is abundant in the tumour microenvironment with the co-stimulatory signal of a second TNFR which is typically provided only in a tightly regulated manner. For example, 4-1BB/4-1BBL and OX40/OX40L are both expressed during a short period after activation on CD8+ and CD4+ T-cells/antigen presenting cells only if antigen is present. By way of example, in lymphomas CD70 is abundant so a CD27-4-1BB chimera might be expressed; in lytic bone metastasis RANKL is abundant to RANK-4-1BB might be expressed. In solid cancers, TWEAK is abundant so Fn14-4-1BB might be expressed.

Without wishing to be bound by theory, engineered cells which express a chimeric TNFR that transmits survival and/or proliferative signals in response to the presence of a ligand which is present in a tumour microenvironment will have improved engraftment and expansion compared to corresponding engineered cells without the chimeric TNFR.

### TNF Receptors

Members of the TNF receptor superfamily (TNFRSF) are typically Type I transmembrane glycoproteins (N-terminus exterior to the cell). The structural motifs in the cytoplasmic domains of TNF superfamily categorize them into two groups based on their signalling properties: those contain a death domain (DD) and others that engage TNFR-associated factors (TRAFs). There is a third group which lack a membrane-anchor domain and are proteolytically cleaved from the surface, or are anchored via glycolipid linkage and are termed “decoy receptors”.

TNFRs comprise a binding domain which is positioned on the extracellular side of the cell membrane when the TNFR is expressed in a cell and a signalling domain which is expressed on the cytoplasmic side of the cell membrane when the TNFR is expressed in a cell.

TNFRSF9 (4-1BB), TNFRSF4 (OX40), TNFRSF5 (CD40) and TNFRSF14 (GITR) transmit survival signals to T-cells. TNFRSF7 (CD27) and TNFRSF14 (HVEM) are expressed by naïve T-cells. The expression of OX40 and 4-1BB is induced in response to antigen stimulation, these TNFRs have been proposed to be markers of effector T cells. Although CD27 and GITR can be constitutively expressed by conventional T cells, their expression is also strongly upregulated following T-cell activation, possibly in parallel with the upregulation of OX40 and 4-1BB expression.

The induction or upregulation of OX40, 4-1BB and GITR expression occurs within 24 hours following the recognition of antigen by and activation of naive T cells, and much more rapidly by memory T cells; the expression of these receptors can last for several hours or even days.

The TNF receptor TNFRSF35/Death receptor 3 (D3R) is activated by TL1A which is upregulated by inflamed tissue transiently and this interaction appears to be important for the late stage of T-cell activity after an established immune response.

CD40 is not expressed by T-cells, but CD40L is and CD40/CD40L is particularly important for B-cell differentiation and expansion.

TNFRSF11A (RANK) is not expressed by T-cells, but the RANK/RANK-L pathway is important to immune development as well as being a key pathway for osteoclast activity and is active during bone metastasis.

TNFRSF12A (Fn14) is not expressed by T-cells, but is expressed along with its ligand TWEAK in damaged or inflamed tissues and most cancers.

### TNF Family Ligands

The TNF-related cytokines (TNF family ligands) are type II transmembrane proteins (intracellular N-terminus) with a short cytoplasmic tail (15 to 25 residues in length) and a larger extracellular region (~50 amino acids) containing the signature TNF homology domain where the receptor binding sites are located.

The TNF homology domain assembles into trimers, the functional unit of the ligand. Atomic analysis of several members of the family revealed that the ligands have a highly conserved tertiary structure folding into a β sheet sandwich, yet amino acid sequence conservation is limited to <35% among the family members.

The conserved residues defining this superfamily are primarily located within the internal β strands that form the molecular scaffold, which promote assembly into trimers. The residues in the loops between the external B-strands are variable and in specific loops make contact with the receptor.

A summary of TNFRs and their ligands is provided in Table 2.

TABLE 2

| Protein (member #) | Synonyms | Gene | Ligand(s) |
|---|---|---|---|
| Tumor necrosis factor | CD120a | TNFRSF1A | TNF-alpha (cachectin) |
| receptor 1 | | | |
| Tumor necrosis factor | CD120b | TNFRSF1B | TNF-alpha (cachectin) |
| receptor 2 | | | |
| Lymphotoxin beta | CD18 | LTBR | Lymphotoxin beta (TNF-C) |
| receptor | | | |
| OX40 | CD134 | TNFRSF4 | OX40L |
| CD40 | Bp50 | CD40 | CD154 |
| Fas receptor | Apo-1, CD95 | FAS | FasL |
| Decoy receptor 3 | TR6, M68 | TNFRSF6B | FasL, LIGHT, TL1A |
| CD27 | S152, Tp55 | CD27 | CD70, Siva |
| CD30 | Ki-1 | TNFRSF8 | CD153 |
| 4-1BB | CD137 | TNFRSF9 | 4-1BB ligand |
| Death receptor 4 | TRAILR1, Apo-2, | TNFRSF10A | TRAIL |
| | CD261 | | |
| Death receptor 5 | TRAILR2, CD262 | TNFRSF10B | TRAIL |
| Decoy receptor 1 | TRAILR3, LIT, TRID, | TNFRSF10C | TRAIL |
| | CD263 | | |
| Decoy receptor 2 | TRAILR4, TRUNDD, | TNFRSF10D | TRAIL |
| | CD264 | | |
| RANK | CD265 | TNFRSF11A | RANKL |
| Osteoprotegerin | OCIF, TR1 | TNFRSF11B | |
| TWEAK receptor | Fn14, CD266 | TNFRSF12A | TWEAK |
| TACI | IGAD2, CD267 | TNFRSF13B | APRIL, BAFF, CAMLG |
| BAFF receptor | CD268 | TNFRSF13C | BAFF |
| Herpesvirus entry | ATAR, TR2, CD270 | TNFRSF14 | LIGHT |
| mediator | | | |
| Nerve growth factor receptor | p75NTR, CD271 | NGFR | NGF, BDNF, NT-3, NT-4 |
| B-cell maturation antigen | TNFRSF13A, CD269 | TNFRSF17 | BAFF |
| Glucocorticoid-induced | AITR, CD357 | TNFRSF18 | GITR ligand |
| TNFR-related | | | |
| TROY | TAJ, TRADE | TNFRSF19 | unknown |
| Death receptor 6 | CD358 | TNFRSF21 | |
| Death receptor 3 | Apo-3, TRAMP, LARD, WS-1 | TNFRSF25 | TL1A |
| Ectodysplasin A2 | XEDAR | EDA2R | EDA-A2 |
| receptor | | | |

### TNFR Binding Domain

The chimeric TNFR binding domain may be any domain which is capable of binding to a TNFR ligand. The binding domain may, for example, comprise the ligand binding domain of a TNFR or an antibody or a part thereof which is capable of binding to a TNFR ligand.

The binding domain may be capable of binding to a TNFR ligand as listed in Table 2. The binding domain may be capable of binding to a TNFR ligand which is present in a tumour microenvironment.

The binding domain may be capable of binding to a TNFR ligand selected from CD70, Receptor activator of NFκB (RANKL), TNF-related weak induce of apoptosis (TWEAK), TNFSF14 (LIGHT), TNFSF1 (LTA), BTLA (CD272), CD160, OX40L, 4-1BBL, CD30L, GITRL, TL1A and CD40L.

The binding domain may be capable of binding to CD70.

The binding domain may be capable of binding to RANKL.

The binding domain may be capable of binding to TWEAK.

The binding domain may be capable of binding to LIGHT, LTA, BTLA and/or CD160.

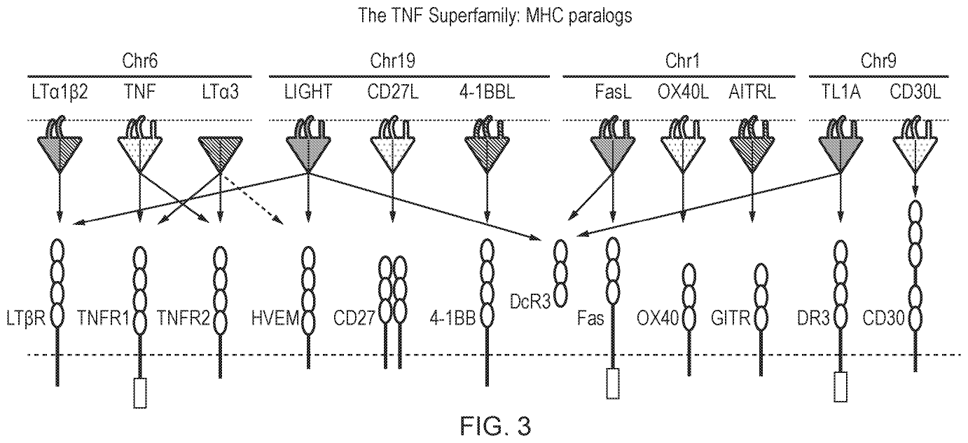

Suitably, the binding domain may comprise the ligand binding domain of a TNFR that binds to the TNFR ligand. A summary of TNFR/TNFR ligand interactions is provided in Table 2 and FIG. 3.

The binding domain may comprise the ligand binding domain selected from CD27, RANKL, Fn14, HVEM, OX40, 4-1BB, CD30, DR3, GITR or CD40 ligand binding domains.

The binding domain may comprise the ligand binding domain of CD27. CD27 expression is tightly controlled during early T-cell differentiation. CD70, the ligand of CD27, is widely expressed on activated T-cells, B-cells and macrophages. CD70 is also expressed on malignant cells such as B-cell lymphomas, leukaemia and gliomas and several other tumours.

An illustrative ligand binding domain of CD27 is shown as SEQ ID NO: 7. Suitably the ligand binding domain of CD27 may comprise SEQ ID NO: 7 or a variant thereof which has the ability to bind CD70. The SEQ ID NO: 7 variant may have at least 80, 85, 90, 95, 98 or 99% sequence identity to SEQ ID NO: 7 and have equivalent or improved CD70 binding capabilities as the sequence shown as SEQ ID NO: 7.

SEQ ID NO: 7
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIR

The binding domain may comprise the ligand binding domain of RANK. The RANK system serves an important role in the immune system, including in lymph-node development, lymphocyte differentiation, dendritic cell survival and T-cell activation, and tolerance induction. RANKL is expressed in several tissues and organs including: skeletal muscle, thymus, liver, colon, small intestine, adrenal gland, osteoblast, mammary gland epithelial cells, prostate and pancreas. A key role of the RANKL/RANK system is mediation of osteoclast-dependent bone remodelling and hence pathologic processes in metastatic disease to bone and is active in all osteolytic bone tumours which includes breast, lung and prostate cancer metastasis to bone and primary marrow disease—namely myeloma. RANK is not normally expressed on, for example, T-cells. Expression of the RANK ligand binding domain on a CAR T-cell, for example, may lead to wide activation particularly at sights of bone-marrow metastasis.

An illustrative ligand binding domain of RANK is shown as SEQ ID NO: 8. Suitably the ligand binding domain of RANK may comprise SEQ ID NO: 8 or a variant thereof which has the ability to bind RANKL. The SEQ ID NO: 8 variant may have at least 80, 85, 90, 95, 98 or 99% sequence identity to SEQ ID NO: 8 and have equivalent or improved RANKL binding capabilities as the sequence shown as SEQ ID NO: 8.

SEQ ID NO: 8
MAPRARRRRPLFALLLLCALLARLQVALQIAPPCTSEKHYEHLGRCCNKC
EPGKYMSSKCTTTSDSVCLPCGPDEYLDSWNEEDKCLLHKVCDTGKALVA
VVAGNSTTPRRCACTAGYHWSQDCECCRRNTECAPGLGAQHPLQLNKDTV
CKPCLAGYFSDAFSSTDKCRPWTNCTFLGKRVEHHGTEKSDAVCSSSLPA
RKPPNEPHVYLP

The binding domain may comprise the ligand binding domain of Fn14. Fn14 can be inducibly expressed on almost all tissues except lymphocytes. The ligand of Fn14 is TWEAK which is upregulated in tissues upon tissue injuries. TWEAK is produced by activated monocytes. TWEAK is also almost universally expressed in tumour microenvironments. Given that T-cells do not express Fn14, they cannot interpret TWEAK in the microenvironment. A chimeric TNFR which is capable of binding TWEAK will transmit activating and survival signals to, for example, a CAR T-cell within the tumour microenvironment.

An illustrative ligand binding domain of Fn14 is shown as SEQ ID NO: 0. Suitably the ligand binding domain of Fn14 may comprise SEQ ID NO: 0 or a variant thereof which has the ability to bind TWEAK. The SEQ ID NO: 9 variant may have at least 80, 85, 90, 95, 98 or 99% sequence identity to SEQ ID NO: 9 and have equivalent or improved TWEAK binding capabilities as the sequence shown as SEQ ID NO: 9.

SEQ ID NO: 9
MARGSLRRLLRLLVLGLWLALLRSVAGEQAPGTAPdwCSRGSSWSADLDK
CMDCASCRARPHSDFCLGCAAAPPAPFRLLWP

The binding domain may comprise the ligand binding domain of HVEM. While most TNF receptors have a single or at most two ligands, HVEM is unique in that it has multiple ligands. These include LIGHT, LTA, BTLA and CD160. Together, these ligands are expressed by a broad range of immune cells. HVEM is normally expressed on naïve and quiescent T-cells but not by activated ones. The expression of a chimeric TNFR comprising a HVEM ligand binding domain allows a CAR T-cell, for example, to receive multiple ligand signals during its activation.

An illustrative ligand binding domain of HVEM is shown as SEQ ID NO: 10. Suitably the ligand binding domain of HVEM may comprise SEQ ID NO: 10 or a variant thereof which has the ability to bind LIGHT, LTA, BTLA and/or CD160. The SEQ ID NO: 10 variant may have at least 80, 85, 90, 95, 98 or 99% sequence identity to SEQ ID NO: 10 and have equivalent or improved LIGHT, LTA, BTLA and CD160 binding capabilities as the sequence shown as SEQ ID NO: 10.

SEQ ID NO: 10
MEPPGDWGPPPWRSTPRTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WV

The binding domain may comprise the ligand binding domain of DR3. DR3 costimulates T-cell activation, and signals through an intracytoplasmic death domain and the adapter protein TRADD (TNFR-associated death domain). TL1A costimulates T cells to produce a wide variety of cytokines and can promote expansion of activated and regulatory T cells in vivo. DR3 also enhances effector T-cell proliferation at the site of tissue inflammation in autoimmune disease models. A chimeric TNFR comprising a DR3 ligand binding domain allows activating and survival signals to be transmitted in, for example, a CAR T-cell within an inflamed tumour microenvironment.

An illustrative ligand binding domain of DR3 is shown as SEQ ID NO: 11. Suitably the ligand binding domain of DR3 may comprise SEQ ID NO: 11 or a variant thereof which has the ability to bind TL1A. The SEQ ID NO: 11 variant may have at least 80, 85, 90, 95, 98 or 99% sequence identity to SEQ ID NO: 11 and have equivalent or improved TL1A binding capabilities as the sequence shown as SEQ ID NO: 11.

SEQ ID NO: 11
MEQRPRGCAAVAAALLLVLLGARAQGGTRSPRCDCAGDFHKKIGLFCCRG
CPAGHYLKAPCTEPCGNSTCLVCPQDTFLAWENHHNSECARCQACDEQAS
QVALENCSAVADTRCGCKPGWFVECQVSQCVSSSPFYCQPCLDCGALHRH
TRLLCSRRDTDCGTCLPGFYEHGDGCVSCPTSTLGSCPERCAAVCGWRQ

As used herein, “antibody” means a polypeptide having an antigen binding site which comprises at least one complementarity determining region (CDR). The antibody may comprise 3 CDRs and have an antigen binding site which is equivalent to that of a domain antibody (dAb). The antibody may comprise 6 CDRs and have an antigen binding site which is equivalent to that of a classical antibody molecule. The remainder of the polypeptide may be any sequence which provides a suitable scaffold for the antigen binding site and displays it in an appropriate manner for it to bind the antigen. The antibody may be a whole immunoglobulin molecule or a part thereof such as a Fab, F (ab)′2, Fv, single chain Fv (ScFv) fragment, Nanobody or single chain variable domain (which may be a VH or VL chain, having 3 CDRs). The antibody may be a bifunctional antibody. The antibody may be non-human, chimeric, humanised or fully human.

The chimeric TNFR binding domain may comprise a binding domain which is not derived from or based on an immunoglobulin. A number of “antibody mimetic” designed repeat proteins (DRPs) have been developed to exploit the binding abilities of non-antibody polypeptides. Such molecules include ankyrin or leucine-rich repeat proteins e.g. DARPins (Designed Ankyrin Repeat Proteins), Anticalins, Avimers and Versabodies.

The binding domain may “specifically bind” to the TNFR ligand as defined herein. As used herein, “specifically bind” means that the binding domain binds to the TNFR ligand but does not bind to other peptides, or binds at a lower affinity to other peptides.

The binding affinity between two molecules, e.g. a TNFR binding domain and a TNFR ligand, may be quantified for example, by determination of the dissociation constant (KD). The KD can be determined by measurement of the kinetics of complex formation and dissociation between the TNFR binding domain and a TNFR ligand, e.g. by the surface plasmon resonance (SPR) method (Biacore™). The rate constants corresponding to the association and the dissociation of a complex are referred to as the association rate constants ka (or kon) and dissociation rate constant kd. (or koff), respectively. KD is related to ka and kd through the equation KD=kd/ka.

Binding affinities associated with different molecular interactions, e.g. comparison of the binding affinity of different TNFR binding domains and a TNFR ligand, may be compared by comparison of the KD values for the individual TNFR binding domain and TNFR ligands.

### TNFR Signalling Domain

The present chimeric TNFR further comprises a TNFR signalling domain.

Suitably, the TNFR signalling domain may be an activating signalling domain. The activating signalling domain may be capable of signalling via TNFR-associated factors (TRAFs).

TRAFs are adaptor proteins that couple TNFRs to signalling pathways. Six members of the TRAF family have been identified (TRAF1-6). All TRAF proteins share a C-terminal homology region termed the TRAF domain that is capable of binding to the cytoplasmic domain of receptors, and to other TRAF proteins. In addition, TRAFs 2-6 have RING and zinc finger motifs that are important for signalling downstream events.

The signalling domain may comprise a domain based on the signalling domain from a TNFR as listed in Table 2.

The signalling domain may comprise a domain based on the signalling domain from a TNFR selected from 4-1BB, OX40, GITR, CD27, CD40, D3R, HVEM, RANK and Fn14.

The signalling domain may comprise a domain based on the signalling domain from a TNFR selected from 4-1BB, OX40, GITR, CD27, CD40, and D3R.

The signalling domain may be based on the signalling domain of 4-1BB. 4-1BB is a co-stimulatory immune checkpoint molecule that provides costimulatory activity for activated T cells. 4-1BB signalling enhances T cell proliferation, IL-2 secretion, survival and cytolytic activity. 4-1BB has been shown to signal via TRAF2.

An illustrative 4-1BB signalling domain is shown as SEQ ID NO: 12. Suitably the 4-1BB signalling domain may comprise SEQ ID NO: 12 or a variant thereof which has the ability to provide a 4-1BB co-stimulatory signal following engagement of the chimeric TNFR binding domain. The SEQ ID NO: 12 variant may have at least 80, 85, 90, 95, 98 or 99% sequence identity to SEQ ID NO: 12 and have equivalent signalling properties to SEQ ID NO: 12.

SEQ ID NO: 12
KRGRKKLLYIFKQPFMRPVQTTQEEDGCSCRFPEEEEGGCEL

The signalling domain may be based on the signalling domain of OX40. OX40 is not constitutively expressed on resting naïve T cells, but is expressed after 24 to 72 hours following activation. OX40 signalling in T cells, for example, is important for T cell survival following activation and for the maintenance of cytokine production. The OX40 signalling domain binds TRAF2, 3 and 5 and PI3 kinase.

An illustrative OX40 signalling domain is shown as SEQ ID NO: 13. Suitably the OX40 signalling domain may comprise SEQ ID NO: 13 or a variant thereof which has the ability to provide a OX40 co-stimulatory signal following engagement of the chimeric TNFR binding domain. The SEQ ID NO: 13 variant may have at least 80, 85, 90, 95, 98 or 99% sequence identity to SEQ ID NO: 13 and have equivalent signalling properties to SEQ ID NO: 13.

SEQ ID NO: 13
RDQRLPPDAHKPPGGGSFRTPIQEEQADAHSTLAKI

The signalling domain may be based on the signalling domain of GITR. GITR expression is increased following T cell activation and is involved in the regulation of CD3-driven T-cell activation and programmed cell death. In particular, it is involved in inhibiting the suppressive activity of T-regulatory cells and extending the survival of T-effector cells

An illustrative GITR signalling domain is shown as SEQ ID NO: 14. Suitably the GITR signalling domain may comprise SEQ ID NO: 14 or a variant thereof which has the ability to provide a GITR co-stimulatory signal following engagement of the chimeric TNFR binding domain. The SEQ ID NO: 14 variant may have at least 80, 85, 90, 95, 98 or 99% sequence identity to SEQ ID NO: 14 and have equivalent signalling properties to SEQ ID NO: 14.

SEQ ID NO: 14
QLGLHIWQLRSQCMWPRETQLLLEVPPSTEDARSCQFPEEERGERSAEEK
GRLGDLWV

The signalling domain may be based on the signalling domain of CD27. CD27 is required for generation and long-term maintenance of T cell immunity. It binds to ligand CD70, and plays a key role in regulating B-cell activation and immunoglobulin synthesis. This receptor transduces signals that lead to the activation of NF-κB and MAPK8/JNK. Adaptor proteins TRAF2 and TRAF5 have been shown to mediate the signalling process of this receptor.

An illustrative CD27 signalling domain is shown as SEQ ID NO: 15. Suitably the CD27 signalling domain may comprise SEQ ID NO: 15 or a variant thereof which has the ability to provide a CD27 co-stimulatory signal following engagement of the chimeric TNFR binding domain. The SEQ ID NO: 15 variant may have at least 80, 85, 90, 95, 98 or 99% sequence identity to SEQ ID NO: 15 and have equivalent signalling properties to SEQ ID NO: 15.

SEQ ID NO: 15
QRRKYRSNKGESPVEPAEPCHYSCPREEEGSTIPIQEDYRKPEPACSP

The signalling domain may be based on the signalling domain of CD40. CD40 is a costimulatory protein found on antigen presenting cells and is required for their activation. The binding of CD154 (CD40L) on TH cells to CD40 activates antigen presenting cells and induces a variety of downstream effects. The CD40 signalling domain interacts with TRAF1, TRAF2 and TRAF6.

An illustrative CD40 signalling domain is shown as SEQ ID NO: 16. Suitably the CD40 signalling domain may comprise SEQ ID NO: 16 or a variant thereof which has the ability to provide a CD40 co-stimulatory signal following engagement of the chimeric TNFR binding domain. The SEQ ID NO: 16 variant may have at least 80, 85, 90, 95, 98 or 99% sequence identity to SEQ ID NO: 16 and have equivalent signalling properties to SEQ ID NO: 16.

SEQ ID NO: 16
KKVAKKPTNKAPHPKQEPQEINFPDDLPGSNTAAPVQETLHGCQPVTQED
GKESRISVQERQ

The signalling domain may be based on the signalling domain of D3R.

An illustrative D3R signalling domain is shown as SEQ ID NO: 17. Suitably the D3R signalling domain may comprise SEQ ID NO: 17 or a variant thereof which has the ability to provide a D3R co-stimulatory signal following engagement of the chimeric TNFR binding domain. The SEQ ID NO: 17 variant may have at least 80, 85, 90, 95, 98 or 99% sequence identity to SEQ ID NO: 17 and have equivalent signalling properties to SEQ ID NO: 17.

SEQ ID NO: 17
TYTYRHCWPHKPLVTADEAGMEALTPPPATHLSPLDSAHTLLAPPDSSEK
ICTVQLVGNSWTPGYPETQEALCPQVTWSWDQLPSRALGPAAAPTLSPES
PAGSPAMMLQPGPQLYDVMDAVPARRWKEFVRTLGLREAEIEAVEVEIGR
FRDQQYEMLKRWRQQQPAGLGAVYAALERMGLDGCVEDLRSRLQRGP

Assays which may be used to determine that a variant signalling domain as described herein is capable of signalling in the same manner as the parent sequence include standard immunological assays which characterize productive T-cell function. These include, for example, flow-cytometric analysis of differentiation, exhaustion and activation; cytotoxicity assays; proliferation assays; measurement of cytokines released and transcriptional profiling (RNAseq).

Suitably, the signalling domain of the present chimeric TNFR is not capable of providing “signal 1” when expressed in a T cell, for example. Thus, the chimeric TNFR is capable of binding a TNFR ligand, but engagement of the chimeric TNFR does not result in productive signal 1 signalling in the cell. In other words, engagement of the chimeric TNFR is capable of providing co-stimulatory signalling to the cell, but is not sufficient to induce full activation of the engineered cell (as it does not provide signal 1).

Thus, in the cell of the present invention, signal 1 is provided upon engagement of the CAR or transgenic TCR and further co-stimulatory signals are provided upon engagement of the chimeric TNFR.

The signalling domain of the chimeric TNFR may consist essential of or consist of a TNFR signalling domain as described herein.

Suitably, the chimeric TNFR does not comprise a CD3 endodomain. Suitably, the chimeric TNFR does not comprise a CD3zeta endodomain (an illustrative CD3zeta endodomain is shown as SEQ ID NO: 31).

### Transmembrane Domain

The chimeric TNFR further comprises a transmembrane domain that spans the membrane. The transmembrane domain may be any protein structure which is thermodynamically stable in a membrane. This is typically an alpha helix comprising of several hydrophobic residues. The transmembrane domain of any transmembrane protein can be used to supply a transmembrane portion. The presence and span of a transmembrane domain of a protein can be determined by those skilled in the art using the TMHMM algorithm (http://www.cbs.dtu.dk/services/TMHMM-2.0/). Further, given that the transmembrane domain of a protein is a relatively simple structure, i.e a polypeptide sequence predicted to form a hydrophobic alpha helix of sufficient length to span the membrane, an artificially designed TM domain may also be used (U.S. Pat. No. 7,052,906 B1 describes transmembrane components).

The transmembrane domain may comprise a hydrophobic alpha helix. The transmembrane domain may be derived from CD28, which gives good receptor stability. The transmembrane domain may comprise the sequence shown as SEQ ID NO: 18 or a variant thereof having at least 80% sequence identity.

SEQ ID NO: 18
FWVLVVVGGVLACYSLLVTVAFIIFWV

The variant may have at least 80, 85, 90, 95, 98 or 99% sequence identity with SEQ ID NO: 18, provided that the variant sequence retains the capacity to traverse the membrane.

The transmembrane domain may be based on the transmembrane domain from a TNFR, for example a TNFR as described herein. Suitably the transmembrane domain may be based on the same TNFR as the signalling domain present in the chimeric TNFR.

Suitably, the transmembrane domain may comprise any one of SEQ ID NO: 19-24 or a variant thereof having at least 80% sequence identity. The variant may have at least 80, 85, 90, 95, 98 or 99% sequence identity with SEQ ID NO: 19-24, provided that the variant sequence retains the capacity to traverse the membrane.

(4-1BB transmembrane domain)
SEQ ID NO: 19
IISFFLALTSTALLFLLFFLTLRFSVV
(DR3 transmembrane domain)
SEQ ID NO: 20
MFWVQVLLAGLVVPLLLGATL
(OX40 transmembrane domain)
SEQ ID NO: 21
VAAILGLGLVLGLLGPLAILL
(GITR transmembrane domain)
SEQ ID NO: 22
LGWLTVVLLAVAACVLLLTSA
(CD70 transmembrane domain)
SEQ ID NO: 23
VLRAALVPLVAGLVICLVVCI
(CD40 transmembrane domain)
SEQ ID NO: 24
ALVVIPIIFGILFAILLVLVFI

Any combination of a binding domain which binds a TNFR ligand for a first TNFR and a signalling domain from a second TNFR may be appropriate for the present invention.

Without wishing to be bound by theory, the following combination may be considered to have particular benefits for the function of an engineered T-cell, for example. For instance, non-physiological interactions with ligands for HVEM, CD27, RANK and Fn14 are quite distinct from physiological TNF ligand interactions. Regarding signalling domains, 4-1BB is known to provide particularly important co-stimulatory signals for T cell function.

The present chimeric TNFR may comprise a binding domain which binds to a ligand of HVEM, CD27, RANK, DR3 or Fn14 and the 4-1BB signalling domain.

The chimeric TNFR may comprise the HVEM binding domain and the 4-1BB signalling domain. An illustrative HVEM/4-1BB chimeric TNFR is shown as SEQ ID NO: 2. Suitably the HVEM/4-1BB chimeric TNFR may comprise SEQ ID NO: 2 or a variant thereof which has the ability to bind a HVEM ligand and provide a 4-1BB co-stimulatory signal following engagement of the chimeric TNFR binding domain. The SEQ ID NO: 2 variant may have at least 80, 85, 90, 95, 98 or 99% sequence identity to SEQ ID NO: 2 and have equivalent signalling properties to SEQ ID NO: 2.

SEQ ID NO: 2
MEPPGDWGPPPWRSTPRTDVLRLVLYLTFLGAPCYAPALPSCKEDEYPVG
SECCPKCSPGYRVKEACGELTGTVCEPCPPGTYIAHLNGLSKCLQCQMCD
PAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSH
WVIISFFLALTSTALLFLLFFLTLRFSVVKRGRKKLLYIFKOPFMRPVQT
TQEEDGCSCRFPEEEEGGCEL

HVEM ectodomain is in normal text (Uniprot; Q92956)
4-1BB transmembrane domain is in bold (Uniprot; Q07011)
4-1BB endodomain is in italics (Uniprot; Q07011)

The chimeric TNFR may comprise the CD27 ligand binding domain and the 4-1BB signalling domain. An illustrative HVEM/4-1BB chimeric TNFR is shown as SEQ ID NO: 3. Suitably the CD27/4-1BB chimeric TNFR may comprise SEQ ID NO: 3 or a variant thereof which has the ability to bind a CD27 ligand and provide a 4-1BB co-stimulatory signal following engagement of the chimeric TNFR binding domain. The SEQ ID NO: 3 variant may have at least 80, 85, 90, 95, 98 or 99% sequence identity to SEQ ID NO: 3 and have equivalent signalling properties to SEQ ID NO: 3.

SEQ ID NO: 3
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRIISFFLALT
STALLFLLFFLTLRFSVVKRGRKKLLYIFKOPFMRPVQTTQEEDGCSCRF
PEEEEGGCEL

CD27 ectodomain is in normal text (Uniprot; P26842)
4-1BB transmembrane domain is in bold (Uniprot; Q07011)
4-1BB endodomain is in italics (Uniprot; Q07011)

The chimeric TNFR may comprise the RANK ligand binding domain and the 4-1BB signalling domain. An illustrative RANK/4-1BB chimeric TNFR is shown as SEQ ID NO: 4. Suitably the RANK/4-1BB chimeric TNFR may comprise SEQ ID NO: 4 or a variant thereof which has the ability to bind a RANK ligand and provide a 4-1BB co-stimulatory signal following engagement of the chimeric TNFR binding domain. The SEQ ID NO: 4 variant may have at least 80, 85, 90, 95, 98 or 99% sequence identity to SEQ ID NO: 4 and have equivalent signalling properties to SEQ ID NO: 4.

SEQ ID NO: 4
MAPRARRRRPLFALLLLCALLARLQVALQIAPPCTSEKHYEHLGRCCNKC
EPGKYMSSKCTTTSDSVCLPCGPDEYLDSWNEEDKCLLHKVCDTGKALVA
VVAGNSTTPRRCACTAGYHWSQDCECCRRNTECAPGLGAQHPLQLNKDTV
CKPCLAGYFSDAFSSTDKCRPWTNCTFLGKRVEHHGTEKSDAVCSSSLPA
RKPPNEPHVYLPIISFFLALTSTALLFLLFFLTLRFSVVKRGRKKLLYIF
KQPFMRPVQTTQEEDGCSCRFPEEEEGGCEL

RANK ectodomain is in normal text (Uniprot; Q9Y6Q6)
4-1BB transmembrane domain is in bold (Uniprot; Q07011)
4-1BB endodomain is in italics (Uniprot; Q07011)

The chimeric TNFR may comprise the Fn14 ligand binding domain and the 4-1BB signalling domain. An illustrative Fn14/4-1BB chimeric TNFR is shown as SEQ ID NO: 5. Suitably the Fn14/4-1BB chimeric TNFR may comprise SEQ ID NO: 5 or a variant thereof which has the ability to bind a Fn14 ligand and provide a 4-1BB co-stimulatory signal following engagement of the chimeric TNFR binding domain. The SEQ ID NO: 5 variant may have at least 80, 85, 90, 95, 98 or 99% sequence identity to SEQ ID NO: 5 and have equivalent signalling properties to SEQ ID NO: 5.

SEQ ID NO: 5
MARGSLRRLLRLLVLGLWLALLRSVAGEQAPGTAPdwCSRGSSWSADLDK
CMDCASCRARPHSDFCLGCAAAPPAPFRLLWPIISFFLALTSTALLFLLF
FLTLRFSVVKRGRKKLLYIFKOPFMRPVOTTQEEDGCSCRFPEEEEGGCE
L

Fn14 ectodomain is in normal text (Uniprot; Q9NP84)
4-1BB transmembrane domain is in bold (Uniprot; Q07011)
4-1BB endodomain is in italics (Uniprot; Q07011)

The chimeric TNFR may comprise the D3R ligand binding domain and the 4-1BB signalling domain. An illustrative D3R/4-1BB chimeric TNFR is shown as SEQ ID NO: 1. Suitably the D3R/4-1BB chimeric TNFR may comprise SEQ ID NO: 1 or a variant thereof which has the ability to bind a D3R ligand and provide a 4-1BB co-stimulatory signal following engagement of the chimeric TNFR binding domain. The SEQ ID NO: 1 variant may have at least 80, 85, 90, 95, 98 or 99% sequence identity to SEQ ID NO: 1 and have equivalent signalling properties to SEQ ID NO: 1.

SEQ ID NO: 1
MEQRPRGCAAVAAALLLVLLGARAQGGTRSPRCDCAGDFHKKIGLFCCRG
CPAGHYLKAPCTEPCGNSTCLVCPQDTFLAWENHHNSECARCQACDEQAS
QVALENCSAVADTRCGCKPGWFVECQVSQCVSSSPFYCQPCLDCGALHRH
TRLLCSRRDTDCGTCLPGFYEHGDGCVSCPTSTLGSCPERCAAVCGWRQI
ISFFLALTSTALLFLLFFLTLRFSVVKRGRKKLLYIFKOPFMRPVQTTQE
EDGCSCRFPEEEEGGCEL

TNFRSF25 (D3R) ectodomain is in normal text (Uniprot; Q93038)
4-1BB transmembrane domain is in bold (Uniprot; Q07011)
4-1BB endodomain is in italics (Uniprot; Q07011)

The chimeric TNFR may comprise the CD27 binding domain and the DR3 signalling domain. An illustrative CD27/DR3 chimeric TNFR is shown as SEQ ID NO: 6. Suitably the CD27/DR3 chimeric TNFR may comprise SEQ ID NO: 6 or a variant thereof which has the ability to bind a CD27 ligand and provide a DR3 co-stimulatory signal following engagement of the chimeric TNFR binding domain. The SEQ ID NO: 6 variant may have at least 80, 85, 90, 95, 98 or 99% sequence identity to SEQ ID NO: 6 and have equivalent signalling properties to SEQ ID NO: 6.

SEQ ID NO: 6
MARPHPWWLCVLGTLVGLSATPAPKSCPERHYWAQGKLCCQMCEPGTFLV
KDCDQHRKAAQCDPCIPGVSFSPDHHTRPHCESCRHCNSGLLVRNCTITA
NAECACRNGWQCRDKECTECDPLPNPSLTARSSQALSPHPQPTHLPYVSE
MLEARTAGHMQTLADFRQLPARTLSTHWPPQRSLCSSDFIRMFWVQVLLA
GLVVPLLLGATLTYTYRHCWPHKPLVTADEAGMEALTPPPATHLSPLDSA
HTLLAPPDSSEKICTVQLVGNSWTPGYPETQEALCPQVTWSWDQLPSRAL
GPAAAPTLSPESPAGSPAMMLQPGPQLYDVMDAVPARRWKEFVRTLGLRE
AEIEAVEVEIGRFRDQQYEMLKRWRQQQPAGLGAVYAALERMGLDGCVED
LRSRLQRGP

CD27 ectodomain is in normal text (Uniprot; P26842)
TNFRSF25 (D3R) transmembrane domain is in bold text (Uniprot; Q93038)
TNFRSF25 (D3R) endodomain is in italic text (Uniprot; Q93038)

In one aspect, the present invention provides a chimeric TNFR as defined herein.

Suitably, the cell of the invention may comprise a plurality of different chimeric TNFRs as provided by the present invention. For example, the cell may comprise two, three, four or five different chimeric TNFRs of the invention. By “different TNFRs” it is meant that a first chimeric TNFR may comprise a different ligand binding domain and/or a different signalling domain compared to a second TNFR.

### TNFR Over-Expression

In one aspect the present invention provides a cell which comprises; (i) a chimeric antigen receptor (CAR) or a transgenic T-cell receptor (TCR); and (ii) an exogenous polynucleotide expressing a TNF receptor (TNFR) which is capable of providing a co-stimulatory to the cell following binding of a ligand to the TNFR ligand binding domain.

As used herein, the term “exogenous polynucleotide” means that the polynucleotide which expresses the TNFR is not part of the endogenous genome of the cell. For example, the exogenous polynucleotide may be an engineered nucleic acid construct or a vector.

The exogenous polynucleotide may express CD27, CD40, D3R, HVEM, RANK or Fn14. In one embodiment the exogenous polynucleotide may express CD27 or CD40.

The cell may be any cell as defined herein. Suitably, the cell may be a T-cell, a natural killer (NK) cell or a cytokine induced killer cell.

The ligands for CD27, CD40, D3R, HVEM, RANK and Fn14 are typically present in the inflammatory tumour microenvironment. However, these TNFRs are either expressed by—for example—T cells in a tightly regulated manner (e.g. during particular periods of T cell development) or not generally expressed by T cells.

Accordingly, exogenous expression of CD27, CD40, D3R, HVEM, RANK or Fn14 in a T cell, for example, may provide similar advantages to the chimeric TNFR of the present invention. For example, following engagement by the TNFR ligand which typically binds to the TNFR, the TNFR expressed from the exogenous polynucleotide is capable of transmitting a co-stimulatory signal. This enables the tight temporal and/or spatial control of TNFR signalling to be decoupled in order to provide improved survival signals for engineered cells, for example CAR T cells.

By way of example, in lymphomas CD70 is abundant so a cell over-expressing CD27 may be provided; in lytic bone metastasis RANKL is abundant so a cell over-expressing RANK might be provided. In solid cancers, TWEAK is abundant so a cell over-expressing may be provided.

As used herein, “over-expressed” or “over-expression” refers to an increased level of expression of the TNFR compared to a corresponding, unmodified cell which does not comprise an exogenous polynucleotide which is capable of expressing the TNFR. Expression levels may be determined by methods which are known in the art, for example, real-time quantitative PCR, western blot and/or flow cytometry,

The cell of invention may express at least 1.5-, at least 2-, at least 5-, at least 5-, at least 10-, at least 20-, at least 50-, or at least 100-fold greater levels of the TNFR compared to a corresponding, unmodified cell.

Suitably, the TNFR expressed by the exogenous polynucleotide may not be detectable in a corresponding, unmodified cell.

Illustrative amino acid sequences for CD27, CD40, D3R, HVEM, RANK and Fn14 are shown as SEQ ID NO: 25-30. The exogenous polynucleotide sequence may be capable of expressing a polypeptide comprising the sequence shown as one of SEQ ID NO: 25-30 or a variant thereof which has at least 80% sequence identity to one of SEQ ID NO: 25-30.

The variant may have at least 80, 85, 90, 95, 98 or 99% sequence identity to SEQ ID NO: 25-30 and have equivalent ligand binding and signalling properties to the corresponding parent SEQ ID NO: 25-30.

The variant may have at least 80, 85, 90, 95, 98 or 99% sequence identity to SEQ ID NO: 25 and have equivalent ligand binding and signalling properties to the corresponding parent SEQ ID NO: 25.

The variant may have at least 80, 85, 90, 95, 98 or 99% sequence identity to SEQ ID NO: 26) and have equivalent ligand binding and signalling properties to the corresponding parent SEQ ID NO: 26.

(CD27)
SEQ ID NO: 25
MPEEGSGCSVRRRPYGCVLRAALVPLVAGLVICLVVCIQRFAQAQQQLPLESLGWDVAELQLNHTGPQQDPRLYW
QGGPALGRSFLHGPELDKGQLRIHRDGIYMVHIQVTLAICSSTTASRHHPTTLAVGICSPASRSISLLRLSFHQG
CTIASQRLTPLARGDTLCTNLTGTLLPSRNTDETFFGVQWVRP
(CD40)
SEQ ID NO: 26
MVRLPLQCVLWGCLLTAVHPEPPTACREKQYLINSQCCSLCQPGQKLVSDCTEFTETECLPCGESEFLDTWNRET
HCHQHKYCDPNLGLRVQQKGTSETDTICTCEEGWHCTSEACESCVLHRSCSPGFGVKQIATGVSDTICEPCPVGF
FSNVSSAFEKCHPWTSCETKDLVVQQAGTNKTDVVCGPQDRLRALVVIPIIFGILFAILLVLVFIKKVAKKPTNK
APHPKQEPQEINFPDDLPGSNTAAPVQETLHGCQPVTQEDGKESRISVQERQ
(DR3)
SEQ ID NO: 27
MEQRPRGCAAVAAALLLVLLGARAQGGTRSPRCDCAGDFHKKIGLFCCRGCPAGHYLKAPCTEPCGNSTCLVCPQ
DTFLAWENHHNSECARCQACDEQASQVALENCSAVADTRCGCKPGWFVECQVSQCVSSSPFYCQPCLDCGALHRH
TRLLCSRRDTDCGTCLPGFYEHGDGCVSCPTSTLGSCPERCAAVCGWRQMFWVQVLLAGLVVPLLLGATLTYTYR
HCWPHKPLVTADEAGMEALTPPPATHLSPLDSAHTLLAPPDSSEKICTVQLVGNSWTPGYPETQEALCPQVTWSW
DQLPSRALGPAAAPTLSPESPAGSPAMMLQPGPQLYDVMDAVPARRWKEFVRTLGLREAEIEAVEVEIGRFRDQQ
YEMLKRWRQQQPAGLGAVYAALERMGLDGCVEDLRSRLQRGP
(HVEM)
SEQ ID NO: 28
MEPPGDWGPPPWRSTPKTDVLRLVLYLTFLGAPCYAPALPSCKEDEYPVGSECCPKCSPGYRVKEACGELTGTVC
EPCPPGTYIAHLNGLSKCLQCQMCDPAMGLRASRNCSRTENAVCGCSPGHFCIVQDGDHCAACRAYATSSPGQRV
QKGGTESQDTLCQNCPPGTFSPNGTLEECQHQTKCSWLVTKAGAGTSSSHWVWWFLSGSLVIVIVCSTVGLIICV
KRRKPRGDVVKVIVSVQRKRQEAEGEATVIEALQAPPDVTTVAVEETIPSFTGRSPNH
(RANK)
SEQ ID NO: 29
MAPRARRRRPLFALLLLCALLARLQVALQIAPPCTSEKHYEHLGRCCNKCEPGKYMSSKCTTTSDSVCLPCGPDE
YLDSWNEEDKCLLHKVCDTGKALVAVVAGNSTTPRRCACTAGYHWSQDCECCRRNTECAPGLGAQHPLQLNKDTV
CKPCLAGYFSDAFSSTDKCRPWTNCTFLGKRVEHHGTEKSDAVCSSSLPARKPPNEPHVYLPGLIILLLFASVAL
VAAIIFGVCYRKKGKALTANLWHWINEACGRLSGDKESSGDSCVSTHTANFGQQGACEGVLLLTLEEKTFPEDMC
YPDQGGVCQGTCVGGGPYAQGEDARMLSLVSKTEIEEDSFRQMPTEDEYMDRPSQPTDQLLFLTEPGSKSTPPFS
EPLEVGENDSLSQCFTGTQSTVGSESCNCTEPLCRTDWTPMSSENYLQKEVDSGHCPHWAASPSPNWADVCTGCR
NPPGEDCEPLVGSPKRGPLPQCAYGMGLPPEEEASRTEARDQPEDGADGRLPSSARAGAGSGSSPGGQSPASGNV
TGNSNSTFISSGQVMNFKGDIIVVYVSQTSQEGAAAAAEPMGRPVQEETLARRDSFAGNGPRFPDPCGGPEGLRE
PEKASRPVQEQGGAKA
(Fn14)
SEQ ID NO: 30
MARGSLRRLLRLLVLGLWLALLRSVAGEQAPGTAPCSRGSSWSADLDKCMDCASCRARPHSDFCLGCAAAPPAPF
RLLWPILGGALSLTFVLGLLSGFLVWRRCRRREKFTTPIEETGGEGCPAVALIQ

The exogenous polynucleotide may be provided as a nucleic acid construct or a vector, for example. Nucleic acid constructs and vectors may be introduced into a cell using methods which are known in the art, for example by viral transduction.

In one aspect the present invention provides a nucleic acid construct which comprises: (i) a first nucleic acid sequence which encodes i) a chimeric antigen receptor (CAR) or a transgenic T-cell receptor (TCR); and (ii) a second nucleic acid sequence which is an exogenous polynucleotide capable of expressing a TNF receptor (TNFR) which is capable of providing a co-stimulatory to the cell following binding of a ligand to the TNFR ligand binding domain.

The exogenous polynucleotide expressing a TNFR may be an exogenous polynucleotide as defined herein.

The present invention further provides a kit comprising: (i) a first nucleic acid sequence which encodes i) a chimeric antigen receptor (CAR) or a transgenic T-cell receptor (TCR); and (ii) a second nucleic acid sequence which is an exogenous polynucleotide capable of expressing a TNF receptor (TNFR) which is capable of providing a co-stimulatory to the cell following binding of a ligand to the TNFR ligand binding domain.

The first nucleic acid sequence and second nucleic acid sequence may be separated by a co-expression site which enables expression of the first nucleic acid sequence which encodes a chimeric antigen receptor (CAR) or a transgenic T-cell receptor (TCR); and the second nucleic acid sequence which is an exogenous polynucleotide capable of expressing a TNF receptor (TNFR) which is capable of providing a co-stimulatory to the cell following binding of a ligand to the TNFR ligand binding domain from a single nucleic acid construct.

Suitably, the co-expression site may be a co-expression site as defined herein.

The present invention also provides a vector, or kit of vectors which comprises one or more nucleic acid sequence(s) or nucleic acid construct(s) which comprises (i) a first nucleic acid sequence which encodes i) a chimeric antigen receptor (CAR) or a transgenic T-cell receptor (TCR); and (ii) a second nucleic acid sequence which is an exogenous polynucleotide capable of expressing a TNF receptor (TNFR) which is capable of providing a co-stimulatory to the cell following binding of a ligand to the TNFR ligand binding domain.

The vector may be a vector as defined herein.

The nucleic acid construct or vector may be for use in therapy as described herein.

### Chimeric Antigen Receptor (CAR)

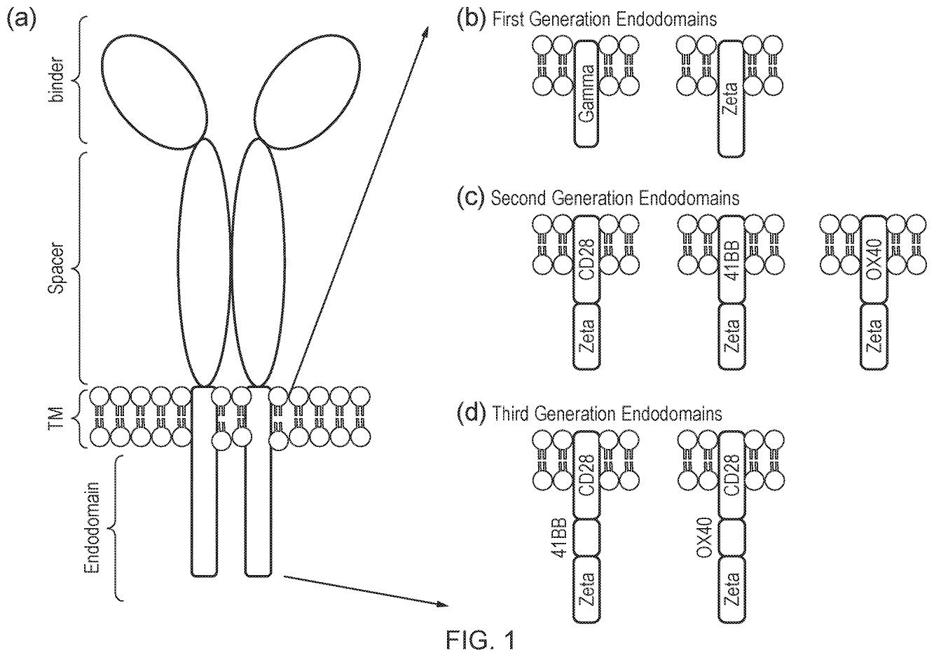

Classical CARs, which are shown schematically in FIG. 1, are chimeric type I trans-membrane proteins which connect an extracellular antigen-recognizing domain (binder) to an intracellular signalling domain (endodomain). The binder is typically a single-chain variable fragment (scFv) derived from a monoclonal antibody (mAb), but it can be based on other formats which comprise an antibody-like antigen binding site or on a ligand for the target antigen. A spacer domain may be necessary to isolate the binder from the membrane and to allow it a suitable orientation. A common spacer domain used is the Fc of IgG1. More compact spacers can suffice e.g. the stalk from CD8a and even just the IgG1 hinge alone, depending on the antigen. A trans-membrane domain anchors the protein in the cell membrane and connects the spacer to the endodomain.

Early CAR designs had endodomains derived from the intracellular parts of either the y chain of the FcεR1 or CD3ζ. Consequently, these first generation receptors transmitted immunological signal 1, which was sufficient to trigger T-cell killing of cognate target cells but failed to fully activate the T-cell to proliferate and survive. To overcome this limitation, compound endodomains have been constructed: fusion of the intracellular part of a T-cell co-stimulatory molecule to that of CD34 results in second generation receptors which can transmit an activating and co-stimulatory signal simultaneously after antigen recognition. The co-stimulatory domain most commonly used is that of CD28. This supplies the most potent co-stimulatory signal—namely immunological signal 2, which triggers T-cell proliferation. Some receptors have also been described which include TNF receptor family endodomains, such as the closely related OX40 and 4-1BB which transmit survival signals. Even more potent third generation CARs have now been described which have endodomains capable of transmitting activation, proliferation and survival signals.

CAR-encoding nucleic acids may be transferred to T cells using, for example, retroviral vectors. In this way, a large number of antigen-specific T cells can be generated for adoptive cell transfer. When the CAR binds the target-antigen, this results in the transmission of an activating signal to the T-cell it is expressed on. Thus the CAR directs the specificity and cytotoxicity of the T cell towards cells expressing the targeted antigen.

### Antigen Binding Domain

The antigen-binding domain is the portion of a classical CAR which recognizes antigen.

Numerous antigen-binding domains are known in the art, including those based on the antigen binding site of an antibody, antibody mimetics, and T-cell receptors. For example, the antigen-binding domain may comprise: a single-chain variable fragment (scFv) derived from a monoclonal antibody; a natural ligand of the target antigen; a peptide with sufficient affinity for the target; a single domain binder such as a camelid; an artificial binder single as a Darpin; or a single-chain derived from a T-cell receptor.

Various tumour associated antigens (TAA) are known, as shown in the following Table 1. The antigen-binding domain used in the present invention may be a domain which is capable of binding a TAA as indicated therein.

TABLE 1

| Cancer type | TAA |
|---|---|
| Diffuse Large B-cell Lymphoma | CD19, CD20 |
| Breast cancer | ErbB2, MUC1 |
| AML | CD13, CD33 |
| Neuroblastoma | GD2, NCAM, ALK, GD2 |
| B-CLL | CD19, CD52, CD160 |
| Colorectal cancer | Folate binding protein, CA-125 |
| Chronic Lymphocytic Leukaemia | CD5, CD19 |
| Glioma | EGFR, Vimentin |
| Multiple myeloma | BCMA, CD138 |
| Renal Cell Carcinoma | Carbonic anhydrase IX, G250 |
| Prostate cancer | PSMA |
| Bowel cancer | A33 |

### Transmembrane Domain

The transmembrane domain is the sequence of a classical CAR that spans the membrane. It may comprise a hydrophobic alpha helix. The transmembrane domain may be derived from CD28, which gives good receptor stability.

### Signal Peptide

The CAR may comprise a signal peptide so that when it is expressed in a cell, such as a T-cell, the nascent protein is directed to the endoplasmic reticulum and subsequently to the cell surface, where it is expressed.

The core of the signal peptide may contain a long stretch of hydrophobic amino acids that has a tendency to form a single alpha-helix. The signal peptide may begin with a short positively charged stretch of amino acids, which helps to enforce proper topology of the polypeptide during translocation. At the end of the signal peptide there is typically a stretch of amino acids that is recognized and cleaved by signal peptidase. Signal peptidase may cleave either during or after completion of translocation to generate a free signal peptide and a mature protein. The free signal peptides are then digested by specific proteases.

### Spacer Domain

The CAR may comprise a spacer sequence to connect the antigen-binding domain with the transmembrane domain. A flexible spacer allows the antigen-binding domain to orient in different directions to facilitate binding.

The spacer sequence may, for example, comprise an IgG1 Fc region, an IgG1 hinge or a human CD8 stalk or the mouse CD8 stalk. The spacer may alternatively comprise an alternative linker sequence which has similar length and/or domain spacing properties as an IgG1 Fc region, an IgG1 hinge or a CD8 stalk. A human IgG1 spacer may be altered to remove Fc binding motifs.

### Intracellular Signalling Domain

The intracellular signalling domain is the signal-transmission portion of a classical CAR.

The most commonly used signalling domain component is that of CD3-zeta endodomain, which contains 3 ITAMs. This transmits an activation signal to the T cell after antigen is bound. CD3-zeta may not provide a fully competent activation signal and additional co-stimulatory signalling may be needed. For example, chimeric CD28 and OX40 can be used with CD3-Zeta to transmit a proliferative/survival signal, or all three can be used together (illustrated in FIG. 1B).

The intracellular signalling domain may be or comprise a T cell signalling domain.

The intracellular signalling domain may comprise one or more immunoreceptor tyrosine-based activation motifs (ITAMs). An ITAM is a conserved sequence of four amino acids that is repeated twice in the cytoplasmic tails of certain cell surface proteins of the immune system. The motif contains a tyrosine separated from a leucine or isoleucine by any two other amino acids, giving the signature YxxL/I. Two of these signatures are typically separated by between 6 and 8 amino acids in the tail of the molecule (YxxL/Ix₍₆₋₈₎ YxxL/I).

ITAMs are important for signal transduction in immune cells. Hence, they are found in the tails of important cell signaling molecules such as the CD3 and ζ-chains of the T cell receptor complex, the CD79 alpha and beta chains of the B cell receptor complex, and certain Fc receptors. The tyrosine residues within these motifs become phosphorylated following interaction of the receptor molecules with their ligands and form docking sites for other proteins involved in the signalling pathways of the cell.

The intracellular signalling domain component may comprise, consist essentially of, or consist of the CD3-ζ endodomain, which contains three ITAMs. Classically, the CD3-ζ endodomain transmits an activation signal to the T cell after antigen is bound. However, in the context of the present invention, the CD3-ζ endodomain transmits an activation signal to the T cell after the MHC/peptide complex comprising the engineered B2M binds to a TCR on a different T cell.

The intracellular signalling domain may comprise additional co-stimulatory signalling. For example, 4-1BB (also known as CD137) can be used with CD3-3, or CD28 and OX40 can be used with CD3-ζ to transmit a proliferative/survival signal.

Accordingly, intracellular signalling domain may comprise the CD3-ζ endodomain alone, the CD3-ζ endodomain in combination with one or more co-stimulatory domains selected from 4-1BB, CD28 or OX40 endodomain, and/or a combination of some or all of 4-1BB, CD28 or OX40.

The endodomain may comprise one or more of the following: an ICOS endodomain, a CD2 endodomain, a CD27 endodomain, or a CD40 endodomain.

The endomain may comprise the sequence shown as SEQ ID NO: 31 to 34 or a variant thereof having at least 80, 85, 90, 95, 98 or 99% sequence identity, provided that the variant sequence retains the capacity to transmit an activating signal to the cell.

The percentage identity between two polypeptide sequences may be readily determined by programs such as BLAST, which is freely available at http://blast.ncbi.nlm.nih.gov. Suitably, the percentage identity is determined across the entirety of the reference and/or the query sequence.

CD3-ζ endodomain
SEQ ID NO: 31
RVKFSRSADAPAYQQGQNQLYNELNLGRREEYDVLDKRRGRDPEMGGKPR
RKNPQEGLYNELQKDKMAEAYSEIGMKGERRRGKGHDGLYQGLSTATKDT
YDALHMQALPPR
4-1BB and CD3-ζ endodomains
SEQ ID NO: 32
MGNSCYNIVATLLLVLNFERTRSLQDPCSNCPAGTFCDNNRNQICSPCPP
NSFSSAGGQRTCDICRQCKGVFRTRKECSSTSNAECDCTPGFHCLGAGCS
MCEQDCKQGQELTKKGCKDCCFGTFNDQKRGICRPWTNCSLDGKSVLVNG
TKERDVVCGPSPADLSPGASSVTPPAPAREPGHSPQIISFFLALTSTALL
FLLFFLTLRFSVVKRGRKKLLYIFKQPFMRPVQTTQEEDGCSCRFPEEEE
GGCELRVKFSRSADAPAYQQGQNQLYNELNLGRREEYDVLDKRRGRDPEM
GGKPQRRKNPQEGLYNELQKDKMAEAYSEIGMKGERRRGKGHDGLYQGLS
TATKDTYDALHMQALPPR
CD28 and CD3-ζ endodomains
SEQ ID NO: 33
SKRSRLLHSDYMNMTPRRPGPTRKHYQPYAPPRDFAAYRSRVKFSRSADA
PAYQQGQNQLYNELNLGRREEYDVLDKRRGRDPEMGGKPRRKNPQEGLYN
ELQKDKMAEAYSEIGMKGERRRGKGHDGLYQGLSTATKDTYDALHMQALP
PR
CD28, OX40 and CD3-ζ endodomains
SEQ ID NO: 34
SKRSRLLHSDYMNMTPRRPGPTRKHYQPYAPPRDFAAYRSRDQRLPPDAH
KPPGGGSFRTPIQEEQADAHSTLAKIRVKFSRSADAPAYQQGQNQLYNEL
NLGRREEYDVLDKRRGRDPEMGGKPRRKNPQEGLYNELQKDKMAEAYSEI
GMKGERRRGKGHDGLYQGLSTATKDTYDALHMQALPPR

Suitably, the CAR may have the general format: antigen-binding domain-TCR element.

As used herein “TCR element” means a domain or portion thereof of a component of the TCR receptor complex. The TCR element may comprise (e.g. have) an extracellular domain and/or a transmembrane domain and/or an intracellular domain e.g. intracellular signalling domain of a TCR element.

The TCR element may selected from TCR alpha chain, TCR beta chain, a CD3 epsilon chain, a CD3 gamma chain, a CD3 delta chain, CD3 epsilon chain.

Suitably, the TCR element may comprise the extracellular domain of the TCR alpha chain, TCR beta chain, a CD3 epsilon chain, a CD3 gamma chain, a CD3 delta chain, or CD3 epsilon chain.

Suitably, the TCR element may comprise the transmembrane domain of the TCR alpha chain, TCR beta chain, a CD3 epsilon chain, a CD3 gamma chain, a CD3 delta chain, or CD3 epsilon chain.

Suitably, the TCR element may comprise the intracellular domain of the TCR alpha chain, TCR beta chain, a CD3 epsilon chain, a CD3 gamma chain, a CD3 delta chain, or CD3 epsilon chain.

Suitably, the TCR element may comprise the TCR alpha chain, TCR beta chain, a CD3 epsilon chain, a CD3 gamma chain, a CD3 delta chain, or CD3 epsilon chain.

### Transgenic T-Cell Receptor (TCR)

The T-cell receptor (TCR) is a molecule found on the surface of T cells which is responsible for recognizing fragments of antigen as peptides bound to major histocompatibility complex (MHC) molecules.

The TCR is a heterodimer composed of two different protein chains. In humans, in 95% of T cells the TCR consists of an alpha (α) chain and a beta (β) chain (encoded by TRA and TRB, respectively), whereas in 5% of T cells the TCR consists of gamma and delta (γ/δ) chains (encoded by TRG and TRD, respectively).

When the TCR engages with antigenic peptide and MHC (peptide/MHC), the T lymphocyte is activated through signal transduction.

In contrast to conventional antibody-directed target antigens, antigens recognized by the TCR can include the entire array of potential intracellular proteins, which are processed and delivered to the cell surface as a peptide/MHC complex.

It is possible to engineer cells to express heterologous (i.e. non-native) TCR molecules by artificially introducing the TRA and TRB genes; or TRG and TRD genes into the cell using a vector. For example the genes for engineered TCRs may be reintroduced into autologous T cells and transferred back into patients for T cell adoptive therapies. Such ‘heterologous’ TCRs may also be referred to herein as ‘transgenic TCRs’.

### Cell

The cell of the present invention may be an immune effector cell, such as a T-cell, a natural killer (NK) cell or a cytokine induced killer cell.

The T cell may be an alpha-beta T cell or a gamma-delta T cell.

The cell may be derived from a patient's own peripheral blood (1st party), or in the setting of a haematopoietic stem cell transplant from donor peripheral blood (2nd party), or peripheral blood from an unconnected donor (3rd party). T or NK cells, for example, may be activated and/or expanded prior to being transduced with nucleic acid molecule(s) encoding the polypeptides of the invention, for example by treatment with an anti-CD3 monoclonal antibody.

Alternatively, the cell may be derived from ex vivo differentiation of inducible progenitor cells or embryonic progenitor cells to T cells. Alternatively, an immortalized T-cell line which retains its lytic function may be used.

The cell may be a haematopoietic stem cell (HSC). HSCs can be obtained for transplant from the bone marrow of a suitably matched donor, by leukopheresis of peripheral blood after mobilization by administration of pharmacological doses of cytokines such as G-CSF [peripheral blood stem cells (PBSCs)], or from the umbilical cord blood (UCB) collected from the placenta after delivery. The marrow, PBSCs, or UCB may be transplanted without processing, or the HSCs may be enriched by immune selection with a monoclonal antibody to the CD34 surface antigen.

The cell of the present invention is an engineered cell. Accordingly, the CAR or transgenic TCR and the chimeric TNFR are not naturally expressed by a corresponding, unmodified cell—for example an unmodified alpha-beta T cell, a NK cell, a gamma-delta T cell or cytokine-induced killer cell.

### Nucleic Acid Construct/Kit of Nucleic Acid Sequences

The present invention provides a nucleic acid construct which comprises: (i) a first nucleic acid sequence which encodes i) a chimeric antigen receptor (CAR) or a transgenic T-cell receptor (TCR); and (ii) a second nucleic acid sequence which encodes a chimeric TNF receptor (TNFR) which comprises (a) a binding domain which is capable of binding a TNFR ligand; and (b) a TNFR activating signalling domain.

Suitably, the chimeric TNF receptor (TNFR) which comprises (a) a binding domain which is capable of binding a TNFR ligand; and (b) a TNFR activating signalling domain may be encoded on a single nucleic acid sequence.

The present invention further provides a kit comprising nucleic acid sequences according to the present invention. For example, the kit may comprise (i) a first nucleic acid sequence which encodes i) a chimeric antigen receptor (CAR) or a transgenic T-cell receptor (TCR); and (ii) a second nucleic acid sequence which encodes a chimeric TNF receptor (TNFR) which comprises (a) a binding domain which is capable of binding a TNFR ligand; and (b) a TNFR activating signalling domain according to the present invention.

As used herein, the terms “polynucleotide”, “nucleotide”, and “nucleic acid” are intended to be synonymous with each other.

Suitably, the nucleic acid construct may comprise a plurality of nucleic acid sequences which encode different chimeric TNFRs as provided by the present invention. For example, the nucleic acid construct may comprise two, three, four or five nucleic acid sequences which different chimeric TNFRs of the invention. Suitably, the plurality of nucleic acid sequences may be separated by co-expression sites as defined herein.

It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described herein to reflect the codon usage of any particular host organism in which the polypeptides are to be expressed. Suitably, the polynucleotides of the present invention are codon optimised to enable expression in a mammalian cell, in particular an immune effector cell as described herein.

Nucleic acids according to the invention may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the use as described herein, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

### Co-Expression Site

A co-expression site is used herein to refer to a nucleic acid sequence enabling co-expression of both i) a chimeric antigen receptor (CAR) or a transgenic T-cell receptor (TCR); and (ii) a chimeric TNF receptor (TNFR) which comprises (a) a binding domain which is capable of binding a TNFR ligand; and (b) a TNFR activating signalling domain as defined herein.

The co-expression site may be a sequence encoding a cleavage site, such that the engineered polynucleotide encodes the enzymes of the transgenic synthetic biology pathway joined by a cleavage site(s). Typically, a co-expression site is located between adjacent polynucleotide sequences which encode separate enzymes of the transgenic synthetic biology pathway.

Suitably, in embodiments where a plurality of co-expression sites is present in the engineered polynucleotide, the same co-expression site may be used.

Preferably, the co-expression site is a cleavage site. The cleavage site may be any sequence which enables the two polypeptides to become separated. The cleavage site may be self-cleaving, such that when the polypeptide is produced, it is immediately cleaved into individual peptides without the need for any external cleavage activity.

The term “cleavage” is used herein for convenience, but the cleavage site may cause the peptides to separate into individual entities by a mechanism other than classical cleavage. For example, for the Foot-and-Mouth disease virus (FMDV) 2A self-cleaving peptide (see below), various models have been proposed for to account for the “cleavage” activity: proteolysis by a host-cell proteinase, autoproteolysis or a translational effect (Donnelly et al (2001) J. Gen. Virol. 82:1027-1041). The exact mechanism of such “cleavage” is not important for the purposes of the present invention, as long as the cleavage site, when positioned between nucleic acid sequences which encode proteins, causes the proteins to be expressed as separate entities.

The cleavage site may be a furin cleavage site. Furin is an enzyme which belongs to the subtilisin-like proprotein convertase family. The members of this family are proprotein convertases that process latent precursor proteins into their biologically active products. Furin is a calcium-dependent serine endoprotease that can efficiently cleave precursor proteins at their paired basic amino acid processing sites. Examples of furin substrates include proparathyroid hormone, transforming growth factor beta 1 precursor, proalbumin, pro-beta-secretase, membrane type-1 matrix metalloproteinase, beta subunit of pro-nerve growth factor and von Willebrand factor. Furin cleaves proteins just downstream of a basic amino acid target sequence (canonically, Arg-X-(Arg/Lys)-Arg′) and is enriched in the Golgi apparatus.

The cleavage site may be a Tobacco Etch Virus (TEV) cleavage site.

TEV protease is a highly sequence-specific cysteine protease which is chymotrypsin-like proteases. It is very specific for its target cleavage site and is therefore frequently used for the controlled cleavage of fusion proteins both in vitro and in vivo. The consensus TEV cleavage site is ENLYFQIS (where ‘1’ denotes the cleaved peptide bond). Mammalian cells, such as human cells, do not express TEV protease. Thus in embodiments in which the present nucleic acid construct comprises a TEV cleavage site and is expressed in a mammalian cell—exogenous TEV protease must also expressed in the mammalian cell.

The cleavage site may encode a self-cleaving peptide. A ‘self-cleaving peptide’ refers to a peptide which functions such that when the polypeptide comprising the proteins and the self-cleaving peptide is produced, it is immediately “cleaved” or separated into distinct and discrete first and second polypeptides without the need for any external cleavage activity.

The self-cleaving peptide may be a 2A self-cleaving peptide from an aphtho- or a cardiovirus. The primary 2A/2B cleavage of the aptho- and cardioviruses is mediated by 2A “cleaving” at its own C-terminus. In apthoviruses, such as foot-and-mouth disease viruses (FMDV) and equine rhinitis A virus, the 2A region is a short section of about 18 amino acids, which, together with the N-terminal residue of protein 2B (a conserved proline residue) represents an autonomous element capable of mediating “cleavage” at its own C-terminus (Donelly et al (2001) as above).

“2A-like” sequences have been found in picornaviruses other than aptho- or cardioviruses, ‘picornavirus-like’ insect viruses, type C rotaviruses and repeated sequences within Trypanosoma spp and a bacterial sequence (Donnelly et al., 2001) as above.

The co-expression sequence may be an internal ribosome entry sequence (IRES). The co-expressing sequence may be an internal promoter.

### Vector

The present invention also provides a vector, or kit of vectors which comprises one or more nucleic acid sequence(s) or nucleic acid construct(s) of the invention. Such a vector may be used to introduce the nucleic acid sequence(s) or construct(s) into a host cell so that it expresses a CAR or transgenic TCR and a chimeric TNF receptor (TNFR) which comprises (a) a binding domain which is capable of binding a TNFR ligand; and (b) a TNFR activating signalling domain.

Suitably, the vector may comprise a plurality of nucleic acid sequences which encode different chimeric TNFRs as provided by the present invention. For example, the vector may comprise two, three, four or five nucleic acid sequences which different chimeric TNFRs of the invention. Suitably, the plurality of nucleic acid sequences may be separated by co-expression sites as defined herein.

The vector may, for example, be a plasmid or a viral vector, such as a retroviral vector or a lentiviral vector, or a transposon based vector or synthetic mRNA.

The vector may be capable of transfecting or transducing a cell.

### Pharmaceutical Composition

The present invention also relates to a pharmaceutical composition containing a cell, a nucleic acid construct, a first nucleic acid sequence and a second nucleic acid sequence; a vector or a first and a second vector of the present invention. In particular, the invention relates to a pharmaceutical composition containing a cell according to the present invention.

The pharmaceutical composition may additionally comprise a pharmaceutically acceptable carrier, diluent or excipient. The pharmaceutical composition may optionally comprise one or more further pharmaceutically active polypeptides and/or compounds. Such a formulation may, for example, be in a form suitable for intravenous infusion.

### Method of Treatment

The present invention provides a method for treating and/or preventing a disease which comprises the step of administering a cell, a nucleic acid construct, a first nucleic acid sequence and a second nucleic acid sequence; a vector or a first and a second vector of the present invention (for example in a pharmaceutical composition as described above) to a subject.

Suitably, the present methods for treating and/or preventing a disease may comprise administering a cell of the invention (for example in a pharmaceutical composition as described above) to a subject.

A method for treating a disease relates to the therapeutic use of the cells of the present invention. In this respect, the cells may be administered to a subject having an existing disease or condition in order to lessen, reduce or improve at least one symptom associated with the disease and/or to slow down, reduce or block the progression of the disease.

The method for preventing a disease relates to the prophylactic use of the cells of the present invention. In this respect, the cells may be administered to a subject who has not yet contracted the disease and/or who is not showing any symptoms of the disease to prevent or impair the cause of the disease or to reduce or prevent development of at least one symptom associated with the disease. The subject may have a predisposition for, or be thought to be at risk of developing, the disease.

The method may involve the steps of:
(i) isolating a cell-containing sample;
(ii) transducing or transfecting such cells with a nucleic acid sequence or vector provided by the present invention;
(iii) administering the cells from (ii) to a subject.

The present invention provides a cell, a nucleic acid construct, a first nucleic acid sequence and a second nucleic acid sequence, a vector, or a first and a second vector of the present invention for use in treating and/or preventing a disease. In particular the present invention provides a cell of the present invention for use in treating and/or preventing a disease

The invention also relates to the use of a cell, a nucleic acid construct, a first nucleic acid sequence and a second nucleic acid sequence, a vector, or a first and a second vector of the present invention of the present invention in the manufacture of a medicament for the treatment and/or prevention of a disease. In particular, the invention relates to the use of a cell in the manufacture of a medicament for the treatment and/or prevention of a disease

The disease to be treated and/or prevented by the method of the present invention may be cancer.

The cancer may be such as bladder cancer, breast cancer, colon cancer, endometrial cancer, kidney cancer (renal cell), leukaemia, lung cancer, melanoma, non-Hodgkin lymphoma, pancreatic cancer, prostate cancer and thyroid cancer.

The cell, in particular the CAR cell, of the present invention may be capable of killing target cells, such as cancer cells. The target cell may be recognisable by expression of a TAA, for example the expression of a TAA provided above in Table 1. The cancer may be a cancer listed in Table 1.

### Method of Making a Cell

CAR or transgenic TCR-expressing cells of the present invention may be generated by introducing DNA or RNA coding for the CAR or TCR and a chimeric TNF receptor (TNFR) which comprises (a) a binding domain which is capable of binding a TNFR ligand; and (b) a TNFR activating signalling domain by one of many means including transduction with a viral vector, transfection with DNA or RNA.

The cell of the invention may be made by:
(i) isolation of a cell-containing sample from a subject or one of the other sources listed above; and
(ii) transduction or transfection of the cells with one or more a nucleic acid sequence(s) or nucleic acid construct as defined above in vitro or ex vivo.

The cells may then by purified, for example, selected on the basis of expression of the antigen-binding domain of the antigen-binding polypeptide.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto.

The invention will now be further described by way of Examples, which are meant to serve to assist one of ordinary skill in the art in carrying out the invention and are not intended in any way to limit the scope of the invention.

### EXAMPLES

### Example 1—In Vitro Testing of a CD27-4-1BB Chimeric TNF Receptor

Primary human T-cells are transduced to express CD19 CAR T-cells and additionally engineered to co-express a CD27-4-1BB chimeric TNF receptor. CD19 CAR T-cells and CD19 CAR T-cells co-expressing CD27-4-1BB are co-cultured with Raji tumour cell lines. Autologous monocytes or dendritic cells which express CD70 are co-cultured with the CAR T-cells/Raji cells. Killing of target cells is determined by flow-cytometry by counting the numbers of surviving target cells. Proliferation, phenotype and of CAR T-cells is determined by flow cytometry.

### Example 2—In Vivo Testing of a CD27-4-1BB Chimeric TNF Receptor

Mice are engrafted with the syngeneic A20 murine lymphoma engineered cell line (engineered to express firefly luciferase) by tail-vein injection. After 10-14 days, the A20 cell line engrafts in the bone-marrow, spleen and lymph node tissues. Murine T-cells are transduced with an anti-(murine)-CD19 CAR or transduced with a bicistronic vector which co-expresses an anti-(murine)-CD19 CAR along with a (murine) CD27-4-1BB chimeric TNF receptor. Three cohorts of A20 burdened mice are given either no CAR T-cells, antiCD19 CAR T-cells or anti-CD19/CD27-4-1BB CAR T-cells. Progression of the A20 tumour is measured by serial bioluminescent imaging. At the end of the experiment, mice are sacrificed, and a necropsy performed. CAR T-cell engraftment is determined by flow-cytometry of blood, marrow, spleen and lymph node tissues. Remaining tumour burden is also determined by flow-cytometry in these tissues as well as immunohistochemistry of bone marrow.

### Example 3—In Vivo Testing of a RANK-4-1BB Chimera

NSG mice receive a tail vein injection of a myeloma cell line (MM1s) engineered to express firefly luciferase. This cell line homes to the bone-marrow of mice and causes lytic bone-lesions. Primary human T-cells are transduced with either a BCMA specific CAR or a cassette which co-expresses the BCMA CAR along with RANK-4-1BB chimeric TNF receptor. After 12 days, mice either receive non-transduced T-cells, BCMA-CAR T-cells or BCMA-CAR/RANK-4-1BB chimera T-cells. Progress of the tumour is determined by serial bioluminescence imaging. Bone lytic lesions are also measured by micro CT. Animals are sacrificed at the end of the experiment. Disease burden is determined by flow-cytometry and immunohistochemistry of bone marrow. Bone lytic lesions can also be measured through histochemistry of bone marrow. T-cell engraftment and persistence are determined by flow cytometry of marrow, spleen, blood and lymphnodes.

### Example 4—In Vitro Testing of a Fn14-4-1BB Chimera

Primary human T-cells are transduced with an EGFRvIII CAR or with a bicistronic vector co-expressing the EGFRvIII CAR and fn14-4-1BB chimeric TNF receptor. Co-cultures with either non-transduced T-cells, CAR T-cells or CAR/fn14-4-1BB T-cells are performed to include a human glioma cell line (such as A172 cell line, the LN-18 cell line or the Ln-229 cell line) engineered to express EGFRvIII. Parallel co-cultures are also performed identically but with the addition of stromal cells which secrete TWEAK. Killing of target cells and proliferation/differentiation of T-cells is determined by flow cytometry.

### Example 5—In Vivo Testing of a Fn14-4-1BB Chimera

Mice are orthotopically engrafted with the murine glioma cell line GL261 expressing murine EGFRvIII using stereotactic injection. Tumour engraftment and progression are determined and measured using micro MRI. Syngeneic splenocytes are transduced with EGFRvIII CAR or a bicistronic vector which co-expresses an EGFRvIII CAR and fn14-4-1BB chimeric TNF receptor. The splenocytes are additionally transduced to express firefly luciferase. GL261 bearing mice are irradiated with low dose (5Gy) total body irradiation and split into three cohorts: No CAR T-cells, EGFRvIII CAR T-cells and EGFRvIII/fn14-4-1BB CAR T-cells. T-cell trafficking, expansion and proliferation is determined through the experiment by serial bioluminescence imaging. Tumour response is determined using micro MRI. At the end of the experiment, the mice are sacrificed. T-cell engraftment systemically is determined by flow-cytometry of bone-marrow, blood, spleen and lymph nodes. Mouse brains are harvested. Half are fixed, and tumour response/T-cell infiltration determined by immunohistochemistry. The other mouse brains are homogenized, and T-cells isolated using Percoll. Isolated T-cell number and quality is determined by flow-cytometry.

### Example 6—In Vitro Testing of RANK-4-1BB or HVEM-4-1BB Chimeric TNF Receptor

Primary human T-cells were transduced to express a first-generation GD2 CAR alone or to co-express GD2 CAR with either a RANK-4-1BB or HVEM-4-1BB chimeric TNF receptor. CAR-T cells were co-cultured with target cells which either expressed the specific tumour antigen (Target cells) or not (Non target cells) in the presence or absence of cells presenting a ligand for the chimeric TNF receptor. Target cell killing was determined by flow-cytometry by counting the numbers of surviving target cells. Proliferation of CAR T-cells was also determined by flow cytometry.

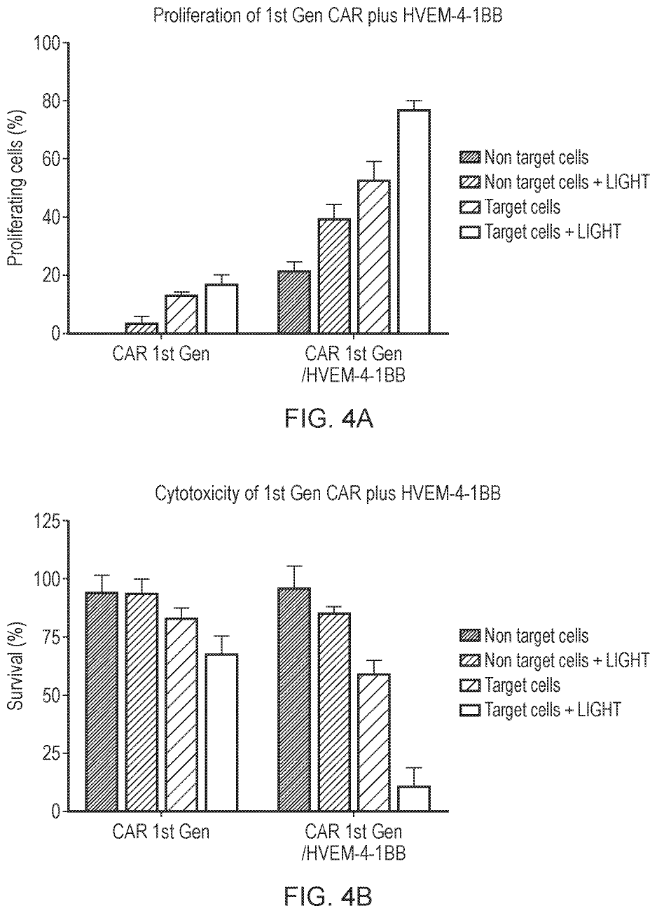

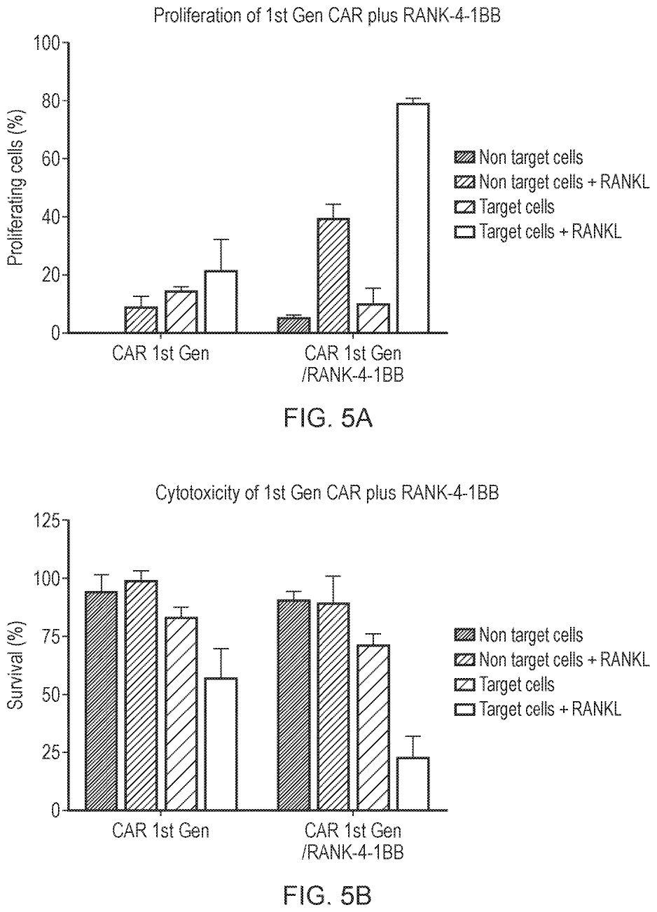

The results are shown in FIGS. 4 and 5. For both the HVEM-4-1BB chimeric TNF receptor (FIG. 4A) and the RANK-4-1BB chimeric TNF receptor (FIG. 5A), expression of the chimeric TNF receptor increased proliferation of T cells, especially in the presence of cells presenting a ligand for the TNF receptor. Expression of the chimeric TNF receptor also increased CAR-mediated target cell killing (FIGS. 4B and 5B) again especially in the presence of cells presenting a ligand for the TNF receptor.

### Cell Culture and Reagents

All cell lines and primary T cells used in the experiments were cultured in RPMI 1640 medium (Lonza) supplemented with 10% fetal bovine serum (FBS, Biosera) and 1% L-Glutamine (GlutaMAX, Gibco). SupT1 cells were purchased from the ATCC. T cells were generated from PBMCs obtained from National Health Service Blood and Transplant (NHSBT; Colindale, UK). Transduced T cells were cultured in the same medium as stated before, with further addition of interleukin-2 (IL-2) at 100 U/mL.

### Retroviral and Plasmid Constructs

Molecular cloning was performed using a mixture of de novo gene synthesis of codon-optimized sequences using overlapping oligonucleotides and cloned into the CAR backbone. Each open reading frame was cloned into the SFG retroviral transfer vector. The TNF-4-1BB sequence was co-expressed by in-frame cloning of the foot- and mouth 2A self-cleaving peptide-based (2A peptide) multi-gene expression system. The RQR8 marker gene was also used, as described in WO2013/153391. RQR8 is recognized by the QBEND/10 anti-CD34 mAb. These markers were introduced into constructs with a codon wobbled 2A peptide in the configuration RQR8_CAR_TNF-4-1BB.

### Transduction

The retrovirus was produced by transient transfection of 293T cells using GeneJuice (Millipore), with a plasmid encoding for gag-pol (pEQ-Pam3-E36), a plasmid encoding for the RD114 envelope (RDF37), and the desired retroviral transfer vector plasmid. Transduction was performed using Retronectin (Takara) as described previously. The transduction efficiency for the different constructs was assessed by flow cytometry based on the expression of RQR8 staining, performed using the QBEND/10 mAb. Flow cytometry analysis was performed using the MACSQuant Analyzer 10 (Miltenyi). Flow sorting was performed using a BD FACS.

### T Cell Proliferation Assay

Cell Trace Violet (CTV) staining was carried out to assess the proliferation of T cells expressing the CAR and the CTNFR sequence (or CAR only) in co-cultures with target cells SupT1-GD2 (or SupT1-NT cells as control), in presence of TNF ligand presenting cells (“RANKL” or “LIGHT”, as found in the figure). T cells expressing the different CAR constructs (NT T cells used as controls) were labelled with CTV before setup of co-cultures with target cells. Staining was performed by re-suspending the T cells at in fresh PBS containing CTV dye, according to the manufacturer's instructions. Co-cultures were at effector: target (E: T) cell ratio of 1:2. Proliferation was assessed by flow cytometry 5 days later. Cells were stained with 7-AAD and CD3 for exclusion of dead cells and detection of T cells, respectively. CTV-stained cells were used to measure proliferation by the extent of dye dilution of dead cells.

### In Vitro Cytotoxicity Assays

T cells expressing the CAR and the TNF-4-1BB proteins (or CAR only) were depleted of CD56-expressing natural killers cells using the EasySep human CD56 positive selection kit (STEMCELL Technologies) according to the manufacturer's instructions. Cells were then used in cytotoxicity assays after 5 and 7 days. Cytotoxicity assays were set up at a 1:2 effector: target (E: T) cell ratio using SupT1 expressing the target in presence/absence of TNF ligand presenting cells (“RANKL” or “LIGHT”, as found in the figure). SupT1 WT cells were used in the same conditions as control. Non transduced (NT) T cells were used in co-cultures with targets as a negative control. CAR-mediated cytotoxicity was assessed by flow cytometry after 5 days. T cells were identified from target cells by CD3 and staining. 7-AAD viability dye was used for exclusion of dead cells. Viable target cells were enumerated for each co-culture condition. The percentage of remaining target cells was calculated by normalizing the number of viable target cells of each condition to that recovered from co-cultures carried out with NT T cells (100%).

All publications mentioned in the above specification are herein incorporated by reference. Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the following claims.

1. A cell which comprises;
(i) a chimeric antigen receptor (CAR) or a transgenic T-cell receptor (TCR); and
(ii) a chimeric TNF receptor (TNFR) which comprises (a) a binding domain which comprises the ligand-binding domain of a first TNFR, wherein the first TNFR is D3R, CD27 or Fn14; and (b) a TNFR signalling domain of a second TNFR, wherein the second TNFR is 4-1BB, OX40, or GITR.

2-5. (canceled)

6. A cell according to claim 1 wherein the signalling domain comprises the 4-1BB endodomain.

7-9. (canceled)

10. A cell according to claim 1 wherein the chimeric TNFR is selected from D3R-4-1BB (SEQ ID NO: 1), CD27-4-1BB (SEQ ID NO: 3), or Fn14-4-1BB (SEQ ID NO: 5).

11-12. (canceled)

13. A cell according to claim 1 wherein the cell is an alpha-beta T cell, a NK cell, a gamma-delta T cell, or a cytokine induced killer cell.

14-17. (canceled)

18. A nucleic acid construct which comprises:
(i) a first nucleic acid sequence which encodes i) a chimeric antigen receptor (CAR) or a transgenic T-cell receptor (TCR); and
(ii) a second nucleic acid sequence which encodes a chimeric TNF receptor (TNFR) which comprises (a) a binding domain which comprises the ligand-binding domain of a first TNFR, wherein the first TNFR is D3R, CD27 or Fn14; and (b) a TNFR signalling domain of a second TNFR, wherein the second TNFR is 4-1BB, OX40 or GIRT.

19-21. (canceled)

22. A kit of nucleic acid sequences or vectors comprising:
(i) a first nucleic acid sequence which encodes i) a chimeric antigen receptor (CAR) or a transgenic T-cell receptor (TCR); and
(ii) a second nucleic acid sequence which encodes a chimeric TNF receptor (TNFR) which comprises (a) a binding domain which comprises the ligand-binding domain of a first TNFR, wherein the first TNFR is D3R, CD27 or Fn14; and (b) a TNFR signalling domain of a second TNFR, wherein the second TNFR is 4-1BB, OX40 or GITF.

23. A vector which comprises a nucleic acid construct according to claim 18.

24. (canceled)

25. A pharmaceutical composition which comprises a plurality of cells according to claim 1.

26. (canceled)

27. A method for treating and/or preventing a disease, which comprises the step of administering a pharmaceutical composition according to claim 25 to a subject in need thereof.

28-31. (canceled)

32. The method according claim 27, wherein the disease is cancer.

33. A method for making a cell according to claim 1, which comprises the step of introducing into a cell ex vivo:
(i) a first nucleic acid sequence which encodes i) a chimeric antigen receptor (CAR) or a transgenic T-cell receptor (TCR); and
(ii) a second nucleic acid sequence which encodes a chimeric TNF receptor (TNFR) which comprises (a) a binding domain which comprises the ligand-binding domain of a first TNFR, wherein the first TNFR is D3R, CD27 or Fn14; and (b) a TNFR signalling domain of a second TNFR, wherein the second TNFR is 4-1BB, OX40 or GITR.

34. A method according to claim 33, wherein the cell is from a sample isolated from a subject.
